SEQUENCE LISTING (7 sequences)

SEQ ID NO: 1 - DNA, 1945 bp, Artificial
  Construct 1 - artificial nucleic acid for detection of AHL in acell-free expression system
tccctatcagtgatagagattgacatccctatcagtgatagagatactgagcactactagagaaagaggagaaatactagatgaaaaacataaatgccgacgacacatacagaataattaataaaattaaagcttgtagaagcaataatgatattaatcaatgcttatctgatatgactaaaatggtacattgtgaatattatttactcgcgatcatttatcctcattctatggttaaatctgatatttcaatcctagataattaccctaaaaaatggaggcaatattatgatgacgctaatttaataaaatatgatcctatagtagattattctaactccaatcattcaccaattaattggaatatatttgaaaacaatgctgtaaataaaaaatctccaaatgtaattaaagaagcgaaaacatcaggtcttatcactgggtttagtttccctattcatacggctaacaatggcttcggaatgcttagttttgcacattcagaaaaagacaactatatagatagtttatttttacatgcgtgtatgaacataccattaattgttccttctctagttgataattatcgaaaaataaatatagcaaataataaatcaaacaacgatttaaccaaaagagaaaaagaatgtttagcgtgggcatgcgaaggaaaaagctcttgggatatttcaaaaatattaggttgcagtgagcgtactgtcactttccatttaaccaatgcgcaaatgaaactcaatacaacaaaccgctgccaaagtatttctaaagcaattttaacaggagcaattgattgcccatactttaaaaattaataacactgatagtgctagtgtagatcactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacaggtttacgcaagaaaatggtttgttatagtcgaataaatactagagtcacacaggaaagtactagatgcgtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataataatactagagccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 2 - DNA, 938 bp, Artificial
  Construct 2 - artificial nucleic acid for detection of AHL in acell-free expression system
acctgtaggatcgtacaggtttacgcaagaaaatggtttgttatagtcgaataaatactagagaaagaggagaaatactagatgcgtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataataatactagagccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 3 - protein, 8 aa, Artificial
  Example peptide thiolactone sequence
YSTCDFIM

SEQ ID NO: 4 - protein, 8 aa, Artificial
  Example peptide thiolactone sequence
YINCDFLL

SEQ ID NO: 5 - protein, 9 aa, Artificial
  Example peptide thiolactone sequence
GVNACSSLF

SEQ ID NO: 6 - protein, 8 aa, Artificial
  Example peptide thiolactone sequence
YSTCYFIM

SEQ ID NO: 7 - protein, 17 aa, Streptococcus pneumoniae
EMRLSKFFRDFILQRKK

(19) United States
(12) Patent Application Publication
(10) Pub. No.: US 2010/0021942 A1
(43) Pub. Date: Jan. 28, 2010

(54) Cell-free expression system for the detection of bacterial biofilms

(76) Inventors: Paul Freemont, London (GB); Richard Ian Kitney, London (GB)

Correspondence Address: MONTGOMERY, MCCRACKEN, WALKER & RHOADS, LLP, 123 SOUTH BROAD STREET, AVENUE OF THE ARTS, PHILADELPHIA, PA, 19109, US

(21) Appl. No.: 12/220,505

(22) Filed: Jul. 25, 2008

(51) Int. Cl. G01N 33/53; G01N 33/00

(52) U.S. Cl. 435/7.9; 436/63

(57) ABSTRACT

Provided is a cell-free expression system and method for its use for detecting the presence of a bacterial biofilm on a surface, wherein the film comprises a) an exogenous quorum sensing protein or an exogenous nucleic acid sequence coding for a quorum sensing protein capable of selectively binding to a bacterial signaling molecule; and b) an exogenous nucleic acid sequence comprising a promoter operably linked to a nucleic acid sequence coding for a marker protein, wherein the promoter is regulated by the binding of the quorum sensing protein to the bacterial signaling molecule. Further provided is an aerosol formulation, as well as an adhesive bandage, each of which may be used for detecting the presence of a bacterial biofilm on a surface, comprising the disclosed cell free expression system and methods therefor.

### FIELD OF THE INVENTION

The invention relates to cell-free expression systems for detecting bacterial biofilms and methods of using the same. The invention also relates to aerosol formulations and adhesive bandages comprising a cell-free expression system for detecting bacterial biofilms.

### BACKGROUND OF THE INVENTION

One of the major causes of mortality and morbidity amongst patients undergoing treatment in hospitals today is due to nosocomial (hospital acquired) infection. Susceptibility to such infection can be as a result of the primary illness for which the patient was admitted, of immuno-suppressive treatment regimes, or as a consequence of injury resulting in serious skin damage, such as burns. The bacterium to which the highest proportion of cases is attributed is Pseudomonas aeruginosa. It is the epitome of an opportunistic pathogen of humans. The bacterium almost never infects uncompromised tissues, yet there is hardly any tissue that it cannot infect, if the tissue defences are compromised in some manner. Although accounting for a relatively small number of species, it poses a serious threat to human health and is used hereafter as a representative example of an infectious bacterium, and does not in any way limit the scope or extent of the present invention.

Ps. aeruginosa is an opportunistic pathogen that causes urinary tract infections, respiratory system infections, dermatitis, soft tissue infections, bacteraemia and a variety of systemic infections, particularly in victims of severe burns, and in cancer and AIDS patients who are immuno-suppressed. Respiratory infections caused by Ps. aeruginosa occur almost exclusively in individuals with a compromised lower respiratory tract or a compromised systemic defence mechanism. Primary pneumonia occurs in patients with chronic lung disease and congestive heart failure. Bacteraemic pneumonia commonly occurs in neutropenic cancer patients undergoing chemotherapy. Lower respiratory tract colonisation of cystic fibrosis patients by mucoid strains of Ps. aeruginosa is common and difficult, if not impossible, to treat. It causes bacteraemia primarily in immuno-compromised patients. Predisposing conditions include haematologic malignancies, immuno-deficiency relating to AIDS, neutropenia, diabetes mellitus, and severe burns. Most Pseudomonas bacteraemia is acquired in hospitals and nursing homes where it accounts for about 25 percent of all hospital acquired gram-negative bacteraemias.

The bacterium is notorious for its natural resistance to many antibiotics due to the permeability barrier afforded by its outer membrane lipopolysaccharide and is, therefore, a particularly dangerous and dreaded pathogen. Also, its tendency to colonise surfaces in a biofilm form makes the cells impervious to therapeutic concentrations of antibiotics (Shih and Huang, J. Antimicrobial Chemotherapy (2002) 49:309-314) and to host phagocytic cells (Wozniak et al., Proc. Natl. Acad. Sci. USA (2003) 100(13):7907-7912).

A biofilm is a structured community of microorganisms encapsulated within a self-developed polymeric matrix and adherent to a living or inert surface. Biofilms are also often characterized by surface attachment, structural heterogeneity, genetic diversity, complex community interactions, and an extracellular matrix of polymeric substances. Biofilms grow and eventually release cells and other debris into the surrounding environment. Released cells then attach to other parts of the surface, starting the cycle anew.

Biofilms are ubiquitous and nearly every species of microorganism, not only bacteria and archaea, have mechanisms by which they can adhere to surfaces and to each other. Cells contained within biofilms are hard to target with normal antibiotics or antiseptics due to the impermeable nature of the biofilm. Biofilms cause widespread problems in, for example, medicine, being implicated in a large number of human infections.

Biofilms are difficult to eradicate and biofilm formation is thought to play a key role in protecting bacteria from host defences. Bacteria in biofilms often display markedly different phenotypes compared to their free-swimming counterparts. Studies have revealed that Ps. aeruginosa isolated from wounds are able to produce an exopolysaccharide capsule within a few hours of infection, a property that is likely to contribute significantly to successful colonisation (Harrisson-Baestra et. al., Dermatol. Surg. (2003) 29(6):631-635). Since its natural habitat is the soil, living in association with the bacilli, actinomycetes and moulds, it has developed resistance to a variety of their naturally occurring antibiotics. Moreover, Pseudomonas spp. maintain antibiotic resistance plasmids, both Resistance factors (R-factors) and Resistance Transfer Factors (RTFs), and are able to transfer these genes by means of the bacterial processes of transduction and conjugation. Only a few antibiotics are effective against Pseudomonas, including fluoroquinolone, gentamicin and imipenem, and even these antibiotics are not effective against all strains. Combinations of gentamicin and carbenicillin are reportedly effective in patients with acute Ps. aeruginosa infections. The futility of treating Pseudomonas infections with antibiotics is most dramatically illustrated in cystic fibrosis patients, virtually all of whom eventually become infected with a strain that is so resistant it cannot be treated. Biofilm formation increases the virulence of the infection considerably.

Biofilms are formed as a result of cell-to-cell signalling in high concentration populations of bacteria. Bacteria make use of cell signalling molecules to detect the presence of other bacterial cells. This is known as quorum sensing (also known as autoinduction or density-dependent gene regulation) and enables bacteria to monitor their own population density. The cell signalling molecules are diffusible and accumulate in the surrounding environment. Once the concentration of cell signalling molecules is sufficiently high, genes required for the formation of a biofilm (or other genes which are regulated in a population density-dependent manner) are activated. González et al. (Microbiol. Mol. Biol. Rev. (2006) 70(4):859-875) provides a review of quorum sensing and the signalling molecules involved.

A classic example of quorum sensing is the autoinduction of luminescence in the marine bacteria Vibrio fischeri (Fuqua et al.; J. Bacteriol. (1994) 176(2):269-275). The plasma membrane of V. fischeri is permeable to the signalling molecule employed by these bacteria (N-3-(oxohexanoyl)homoserine lactone). The signalling molecule hence accumulates in the growth environment, but at sufficient concentrations (when there is a high enough density of bacteria) the molecule also accumulates intracellularly and activates the transcription of a luminescence gene, lux. V. fisheri is found in sea water and also in a symbiotic relationship with certain marine fishes and squids. Quorum sensing allows V. fisheri to conserve resources by limiting lux expression to host-associated states when concentrations of the bacteria are high enough for the luminescence to be seen by the naked eye.

Manago et al. (Infect. Control. Hosp. Epidemiol. (2006) 27:188-190) describe biofilm formation by methicillin-resistant Staphylococcus aureus (MRSA) strains recovered from patients with nosocomial infections. MSRA exploits a signalling system known as the accessory gene regulator (agr) quorum-sensing system. This system has also been linked to biofilm formation in Staphylococcus aureus, which itself is known to be a cause of pneumonia, meningitis, osteomyelitis endocarditis and septicaemia.

Bacterial cell signalling molecules employed by bacteria in quorum sensing include the homoserine lactones, peptide thiolactone, furanosyl borate diesters and other autoinducers, and cyclic dipeptide.

Biofilms are not only a health hazard in hospitals, but also present problems in drinking water systems, water cooling systems, industrial fluid processing systems and food processing systems. Hence early detection of biofilms would allow measures to be taken to prevent the formation of established biofilms that are hard to remove and pose a serious health threat.

Andersen et al. (Appl. Environ. Microbiol. (2001) 67(2):575-585) describe the detection of bacterial communication using a green fluorescent protein-based n-acyl homoserine lactone (AHL) sensor system. Vibrio fischeri transfected with a mutant green fluorescent protein (GFP) fused to quorum sensing machinery of the bacteria were used to detect the presence of AHL in combination with microscopy. In the presence of AHL molecules, the protein LuxR positively affects the expression of a luxI promoter, which then in turn controls the expression of the mutant GFP marker gene. This system requires the use of live bacteria in addition to the use of microscopy to detect the mutant GFP marker.

International patent application published as WO2007/075595 describes methods for measuring biofilms and biofilm formation using compounds that fluoresce upon contacting certain biofilms. More specifically, the application describes the use of a thioflavin to detect the presence of polysaccharide intercellular adhesion molecule (PIA), which is synthesised by Staphylococcus aureus. This allows the detection of biofilms since fluorescence is increased on contact with the PIA relative to the level of fluorescence produced by the compound alone. Detection of fluorescence requires the use of magnetic resonance spectroscopy (MRS) or imaging (MRI), or gamma imaging such as positron emission tomography (PET) or single-photon emission computed tomography (SPECT).

International patent application published as WO03/095995 describes a method of measuring the development of a biofilm using confocal imaging. This technique requires expensive equipment and the deliberate cultivation of a biofilm and is not suitable for the detection of biofilms in situ.

Other biofilm detection methods include visualisation of the biofilm itself by microscopy, culture detection (for example taking a swab and growing on agar) and the detection of genes responsible for biofilm formation. However, these and other methods suffer in that they fail to deliver a fast response, in situ or otherwise, and sensitivity to the presence of biofilms is low.

Given the difficulty associated in the treatment of infections that involve biofilms, a simple and effective method of detecting the presence of a biofilm on a surface remains to be provided. Preferably the method would give a result in situ within a few hours, would be non-invasive, inexpensive, reliable and would not itself be a possible cause of biofilm formation, as is the case for detection systems using live bacteria.

### SUMMARY OF THE INVENTION

In a first aspect of the invention, there is provided a cell-free expression system for detecting the presence of a bacterial biofilm on a surface, comprising:
a) an exogenous quorum sensing protein or an exogenous nucleic acid sequence coding for a quorum sensing protein capable of selectively binding to a bacterial signalling molecule; and
b) an exogenous nucleic acid sequence comprising a promoter operably linked to a nucleic acid sequence coding for a marker protein, wherein the promoter is regulated by the binding of the quorum sensing protein to the bacterial signalling molecule.

In a second aspect of the invention, there is provided an aerosol formulation for detecting the presence of a bacterial biofilm on a surface, comprising a propellant and cell-free expression system, wherein the cell-free expression system comprises:
a) an exogenous quorum sensing protein or an exogenous nucleic acid sequence coding for a quorum sensing protein capable of selectively binding to a bacterial signalling molecule; and
b) an exogenous nucleic acid sequence comprising a promoter operably linked to a nucleic acid sequence coding for a marker protein, wherein the promoter is regulated by the binding of the quorum sensing protein to the bacterial signalling molecule.

In a third aspect of the invention, there is provided an adhesive bandage for detecting the presence of a bacterial biofilm on a surface, comprising a cell-free expression system, wherein the cell-free expression system comprises:
a) an exogenous quorum sensing protein or an exogenous nucleic acid sequence coding for a quorum sensing protein capable of selectively binding to a bacterial signalling molecule; and
b) an exogenous nucleic acid sequence comprising a promoter operably linked to a nucleic acid sequence coding for a marker protein, wherein the promoter is regulated by the binding of the quorum sensing protein to the bacterial signalling molecule.

In a fourth aspect of the invention, there is provided a method of detecting a bacterial biofilm on a surface comprising applying to the surface the cell-free expression system comprising:
a) an exogenous quorum sensing protein or an exogenous nucleic acid sequence coding for a quorum sensing protein capable of selectively binding to a bacterial signalling molecule; and
b) an exogenous nucleic acid sequence comprising a promoter operably linked to a nucleic acid sequence coding for a marker protein, wherein the promoter is regulated by the binding of the quorum sensing protein to the bacterial signalling molecule.

In an embodiment of the invention, there is provided a method of detecting a bacterial biofilm on a surface comprising applying a cell-free expression system comprising:
a) an exogenous quorum sensing protein or an exogenous nucleic acid sequence coding for a quorum sensing protein capable of selectively binding to a bacterial signalling molecule; and
b) an exogenous nucleic acid sequence comprising a promoter operably linked to a nucleic acid sequence coding for a marker protein, wherein the promoter is regulated by the binding of the quorum sensing protein to the bacterial signalling molecule.

Additional objects, advantages and novel features of the invention will be set forth in part in the description, examples and figures which follow, all of which are intended to be for illustrative purposes only, and not intended in any way to limit the invention, and in part will become apparent to those skilled in the art on examination of the following, or may be learned by practice of the invention.

### BRIEF DESCRIPTION OF FIGURES

The foregoing summary, as well as the following detailed description of the invention, will be better understood when read in conjunction with the appended drawings. It should be understood, however, that the invention is not limited to the precise arrangements and instrumentalities shown.

FIG. 1 is a schematic diagram showing the components of a nucleic acid that can be used in the cell-free expression system of the invention (construct 1). tetR (R0040) represents the a promoter for LuxR. B0034 is a ribosome binding site. LuxR (C0062) is the quorum sensing protein. B0010 is the transcriptional terminator T1 from E. coli. B0012 is the transcriptional terminator TE from coliphageT7. Lux pR (R0062) is the AHL and LuxR regulated promoter from V. fischeri. It contains a left and a right promoter. The right promoter gives weak constitutive expression of downstream genes. This expression is up-regulated by the action of the LuxR activator protein complexed with the signalling molecule, 3-oxo-hexanoyl-HSL. Two molecules of LuxR protein form a complex with two molecules of the signalling compound homoserine lactone (HSL). This complex binds to a palindromic site on the promoter, increasing the rate of transcription. GFP E0040 represents the green fluorescent protein derived from jellyfish Aequeora victoria wild-type GFP (mut3bGFP). Its maximum excitation wavelength is 501 nm and its maximum emission wavelengtt is 511 nm.

FIG. 2 shows the sequence (SEQID No:1) of construct 1 aligned with functional labels.

FIG. 3 shows the nucleotide sequence (SEQID No:1) of construct 1.

FIG. 4 shows a schematic diagram showing how construct 1 detects the presence of a bacterial biofilm. pTet promotes the expression of LuxR protein. This binds to AHL produced by bacteria in the biofilm. The resulting conjugate binds to the pLux (Lux pR) promoter and stimulates expression of GFP.

FIG. 5 is a schematic diagram showing how construct 1 detects the presence of a bacterial biofilm at AHL concentrations from 5 nM to 50 nM. The cell-free chassis is the cell-free expression system.

FIG. 6 is a schematic diagram showing the components of a nucliec acid that can be used in the cell-free expression system of the invention (construct 2). Lux pR (R0062) is the AHL and LuxR regulated promoter from V. fischeri. It contains a left and a right promoter. The right promoter gives weak constitutive expression of downstream genes. This expression is up-regulated by the action of the LuxR activator protein complexed with the signalling molecule, 3-oxo-hexanoyl-HSL. Two molecules of LuxR protein form a complex with two molecules of the signalling compound homoserine lactone (HSL). This complex binds to a palindromic site on the promoter, increasing the rate of transcription. GFP E0040 represents the green fluorescent protein derived from jellyfish Aequeora victoria wild-type GFP (mut3bGFP). Its maximum excitation wavelength is 501 nm and its maximum emission wavelength is 511 nm. B0010 is the transcriptional terminator T1 from E. coli. B0012 is the transcriptional terminator TE from coliphageT7.

FIG. 7 shows the sequence (SEQID No:2) of construct 2 aligned with functional labels.

FIG. 8 shows the nucleotide sequence (SEQID No:2) of construct 2.

FIG. 9 is a schematic diagram showing how construct 2 detects the presence of a bacterial biofilm. Exogenously applied LuxR binds to AHL produced by bacteria in the biofilm. The resulting conjugate binds to the pLux (Lux pR) promoter and stimulates expression of GFP

FIG. 10 is a schematic diagram showing how construct 2 detects the presence of a bacterial biofilm at AHL concentrations from 5 nM to 50 nM. The cell-free chassis is the cell-free expression system.

FIG. 11 shows modelled expression of GFP (GFP expression versus time) in arbitrary units and varying initial concentrations of AHL (arbitrary units) using construct 1. As initial AHL concentration is increased, the level of expression increases according to a point where there is negligible difference between the maximal outputs between adjacent AHL concentrations.

FIG. 12 shows modelled energy depletion (energy vs time) of the cell-free expression system using construct 2. Increasing the initial AHL concentration increases the consumption of energy. More resources (promoters) are needed to “accommodate” the increasing AHL concentration.

FIG. 13 shows modelled GFP expression of construct 1, at various initial AHL concentrations in nM (fluorescence versus time).

FIG. 14 shows modelled GFP expression on construct 2 at various initial AHL concentrations in nM (fluorescence versus time).

FIG. 15 shows GFP fluorescence using construct 1 in E. coli cells and in a cell-free expression system and at varying initial concentrations of AHL.

FIG. 16 shows GFP fluorescence using construct 1 in an E. coli S30 extract-based cell-free expression system.

FIG. 17 shows DNA concentration in vitro: Molecules of GFPmut3b synthesised versus time at varying DNA concentrations using construct 1 (AHL concentration was 50 nM and temperature 25° C.).

FIG. 18 shows DNA concentration in vitro: Molecules of GFPmut3b synthesised after 360 minutes at varying DNA concentrations using construct 1 (AHL concentration was 50 nM and temperature 25° C.).

FIG. 19 shows GFP molecules synthesised vs Time for varying AHL concentrations using construct 1 (temperature was 25° C.).

FIG. 20 shows GFPmut3b molecules synthesised after 360 minutes versus AHL concentrations (temperature was 25° C.).

FIG. 21 is a schematic diagram showing the production of vesicles comprising the cell-free expression system of the invention.

FIG. 22 shows fluorescence of simulated biofilms using construct 1.

FIG. 23 shows GFP fluorescence using construct 1 as applied to agar comprising varying concentrations of AHL.

### DETAILED DESCRIPTION OF EMBODIMENTS OF THE INVENTION

In a first aspect of the invention, there is provided a cell-free expression system for detecting the presence of a bacterial biofilm on a surface, comprising:
a) an exogenous quorum sensing protein or an exogenous nucleic acid sequence coding for a quorum sensing protein capable of selectively binding to a bacterial signalling molecule; and
b) an exogenous nucleic acid sequence comprising a promoter operably linked to a nucleic acid sequence coding for a marker protein, wherein the promoter is regulated by the binding of the quorum sensing protein to the bacterial signalling molecule.

Cell-free expression systems (also know as cell-free chassis) are reviewed in Lingappa et al. (Mt. Sinai J. Med. (2005) 72(3): 141-160) and in Jackson et al. (Brief. Funct. Genomic. Proteomic. (2004) 2(4):308-319). Shimizu et al. (Nat. Biotech. (2001) 19:751-755) demonstrate a cell-free expression system using purified components. The cell-free expression system can be a bacterial cell-free expression system, for example an E. coli ell-free expression system.

Surfaces on which the presence of a bacterial biofilm might be investigated include surfaces of medical devices or the surfaces of food production equipment. Medical devices include devices that are applied onto or inserted into a patient. Such devices include a catheter, a cannula, a scalpel, a needle, a speculum, a stent, an implant or a contact lens. Food production equipment includes equipment use on food production lines. Hence the surface can be in or on machinery or equipment used in a food production line. Other production lines which may comprise suitable surfaces include those production lines used to produce medical or sterile equipment. Indeed the invention could be used wherever or whenever deemed suitable by a skilled person.

The surface can be inert of be part of living tissue. The surface can be an internal surface, or an external surface or a lumen.

Bacterial signalling molecules of this aspect of the invention can include all molecules used by bacteria in quorum sensing that promote the formation of bacterial biofilms. Such molecules include the classes homoserine lactones (HSL), peptide thiolactones, furanosyl borate diesters, cyclic dipeptide and competence stimulating peptides (CPS).

Homoserine lactones include all molecules with the following general formula:

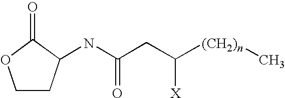

Where X is —H, —OH or ═O and where n=0 to 18. Compounds where X is —H can be described as acyl-homoserine lactones (AHLs). Compounds where X is ═O can be described as 3-oxo-homoserine lactones. Compounds where X is —OH can be described as 3-hydroxy-homoserine lactones. Homoserine lactones are produced by many gram-negative bacteria, for example Vibrio ficheri, Burkholderia cepacia, Legionella family (for example Legionella pneumophila) and Pseudomonas spp..

Specific examples of AHLs include N-butanoyl-L-homoserine lactone (BHL) where n=0 (also known as butyryl homoserine lactone or Pseudomonas aeruginosa autoinducer PAI-2), octanoyl homoserine lactone where n=4 (also known as Vibrio fischeri autoinducer VAI-2), dodecanoyl-L-homoserine lactone where n=8 and n-tetradecanoyl-L-homoserine lactone (tDHL) where n=10.

Specific examples of 3-oxo-homoserine lactones include N-(-3-oxohexanoyl)-L-homoserine lactone (OHHL) where n=2 (also known as Vibrio fischeri autoinducer VAI-1), 3-oxooctanoyl homoserine lactone where n=4 (also known as Agrobacterium tumefaciens autoinducer AAI-1) and N-(-3-oxododecanoyl)-L-homoserine lactone (OdDHL) where n=8.

Specific examples of 3-hydroxy-homoserine lactones include N-(-3-hydroxybutanoyl)-L-homoserine lactone (HBHL) where n=0 (also known as hydroxybutyryl homoserine lactone or Vibrio harveyi autoinducer HAI), or R-hydroxy-7-cis-tetradecenoyl homoserine lactone (also known as Rhizobium leguminosarum autoinducer RLAI).

Peptide thiolactones (also known as small autoinducing peptides or AIPs) can have the general formula:

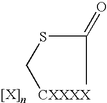

where X is any amino acid and n=1 to 10. Peptide thiolactones are produced by such species as the Staphylococcus family (for example methicillin-resistant Staphylococcus aureus).

Throughout this specification, amino acid residues are designated by the usual International Union of Pure and Applied Chemistry (IUPAC) single letter nomenclature. The single letter designations may be correlated with the classical three letter designations of amino acid residues as follows:

A = Ala
C = Cys
D = Asp
E = Glu
F = Phe
G = Gly
H = His
I = Ile
K = Lys
L = Leu
M = Met
N = Asn
P = Pro
Q = Gln
R = Arg
S = Ser
T = Thr
V = Val
W = Trp
Y = Tyr

Peptide thiolactone may have the following structures:

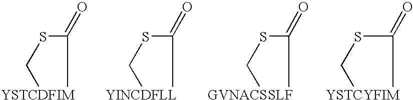

wherein YSTCDFIM is SEQID No:3; YINCDFLL is SEQID No:4; GVNACSSLF is SEQID No:5; and YSTCYFIM is SEQID No:6.

Furanosyl borate diesters can be AutoInducer-2 (AI-2), Pro-AI-2 or Pro-AI-2 reactive hapten. AI-2 can be described as 2,3-dihydroxy-4-methyl-3,4-borate diester and has the formula:

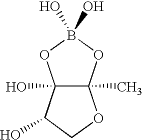

AI-2 is produced by such species as the Salmonella family (for example Salmonella typhimurium) and the Clostridium family (for example Clostridium difficile).

Pro-AI-2 has the following structure:

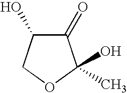

Pro-AI-2 reactive hapten has the following structure:

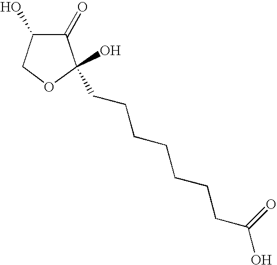

Cyclic dipeptide (also known as diketopiperazines or DKPs) include cyclo(L-Phe-L-Pro) and cyclo(L-Tyr-L-Pro), which have the structure:

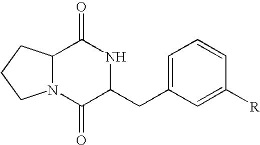

where R═H for cyclo(L-Phe-L-Pro) and R═OH for cyclo(L-Tyr-L-Pro). Cyclic dipeptides are produced by such species as Proteus mirabilis, Citrobacter freundii, Enterobacter agglomerans, Pseudomonas fluorescens, and Pseudomonas alkaligenes.

Competence stimulating peptides (CPS) are small (less than 25 amino acid residues) cationic peptides employed by some bacterial species (for example the Streptococcus family) in cell-to-cell signalling (quorum sensing). An example of a CPS is H-Glu-Met-Arg-Leu-Ser-Lys-Phe-Phe-Arg-Asp-Phe-Ile-Leu-Gln-Arg-Lys-Lys-OH (SEQ ID No:7), as produced by Streptococcus pneumoniae.

Other cell signalling molecules of the invention can include autoinducer AI-3 (identified in Escherichia coli species), bradyoxetin (produced by, for example, Bradyrhizobium japonicum) and diffusible signal factor (or DSF, produced by, for example, Xanthomonas campestris):

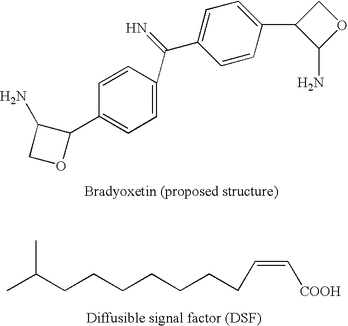

Throughout this specification, the phrases “cell signaling molecules”, “bacterial signalling molecule”, “cell-to-cell signalling molecules”, “signalling molecules” and “autoinducers” all refer to cell signalling molecules produced by bacteria and used in quorum sensing.

Signalling molecules currently known to be involved in bacterial biofilm formation include the homoserine lactones, AI-2, AI-3, competence stimulating peptides and small autoinducing peptides, and others will be apparent to a person of skill in the art.

The bacterial signalling molecules may be produced by either Gram-negative or Gram-positive bacteria, and include Pseudomonas aeruginosa, Burholderia cepacia, Chromobacterium violaceum, Yersinia pestis, Yersinia enterocolitica, Yersinia pseudotuberculosis, Aeromonas hydrophilia, Brucella melitensis, Bacillus subtilis, Streptococcus pneumoniae, Enterococcus faecalis, E. coli, Helicobacter pylori, Neisseria meningitides, Porphyromonas gingivalis, Proteus mirabilis, S. typhimurium, Streptococcus pyrogenese, Stapholococcus aureues et cetera.

Marker proteins of the invention include fluorescent proteins, enzymes and peptide antigens. They may comprise a transcription and/or a translation termination region(s).

Fluorescent proteins include green fluorescent protein (GFP), enhanced green fluorescent protein (EGFP), blue fluorescent protein (BFP), yellow fluorescent protein (YFP), cyan fluorescent protein (CFP), orange fluorescent protein (OFP) or red fluorescent protein (RFP), or derivatives thereof.

Examples of green fluorescent protein derivatives include AcGFP, TurboGFP, Emerald, Azami Green and ZsGreen.

Examples of blue fluorescent protein derivates include enhanced blue fluorescent protein (EBFP), Sapphire and T-Sapphire.

Examples of cyan fluorescent protein derivatives include enhanced cyan fluorescent protein (ECFP), mCFP, Cerulean, CyPet, AmCyanl, Midori-Ishi Cyan and mTHPI (Teal).

Examples of yellow fluorescent protein derivatives include enhanced yellow fluorescent protein (EYFP), Topas, Venus, mCitrine, YPet, PhiYFP, ZsYellow1 and mBanana.

Examples of orange and yellow fluorescent protein derivatives include Kusabira Orange, mOrange, dTomato, dTomato-Tandem, DsRed (Discosoma sp. Red fluorescent protein varient), DsRed2, DsRed-Express (T1), DsRed-Monomer, mTangerine, mStrawberry, AsRed2, mRFP1, JRed, mCherry, HcRed1, mRaspberry, HcRed-Tandem, mPlum and AQ143.

Fluorescent proteins used in a method of the invention may be visible to the naked eye when expressed (for example, DsRed fluorescent protein).

Fluorescent proteins for use in the current invention are available from, for example, Clontech Laboratories, Inc. (Mountain View, Calif., USA) or Promega UK Ltd. (Southampton, United Kingdom).

Enzymes that could be used as a marker protein in the invention include luciferase.

Peptide antigens that could be used as a marker protein in the invention include proteins for which antibodies are commercially available. The presence of the marker protein could be detected using an enzyme-linked immunosorbent assay (ELISA)

The quorum sensing protein that is capable of selectively binding to a bacterial signalling molecule can be selected according to the bacterial signalling molecule being detected. In other words, the respective cognate binding protein of the bacterial signalling molecule being detected is selected as the quorum sensing protein. In turn, the promoter upstream of the marker protein can be selected according to the choice of quorum sensing protein such that the promoter is activated by the binding of the bacterial signalling molecule to the quorum binding protein. In this way, expression of marker protein can be up-regulated. Hence, it can be easily envisaged by a person of skill in the art that the cell-free expression system can be tailored for the detection of different signalling molecules.

Proteins that can are capable of selectively binding to a bacterial signalling molecule include the LuxR family transcriptional regulator (LuxR), the periplasmic AI-2 binding protein LuxP (LuxP), the transcriptional regulator LasR (LasR), the transcription regulator Rh1R (Rh1R), the transcriptional repressor LsrR (LsrR), histidine protein kinase ComD (ComD), the accessory gene regulator AgrC (AgrC), the N-acyl homoserine lactone transcriptional regulator CepR (CepR), the membrane protein LqsS (LqsS), the quorum sensing Escherichia coli regulator C (QseC) or the membrane protein ro1B (ro1B).

LuxR can selectively bind to acyl-homoserine lactone signalling molecules. LuxP can selectively bind to the AI-2 signalling molecule. LasR can selectively bind to lactone signalling molecules, for example N-(3-oxododecanoyl)-L-homoserine lactone and N-butyryl-L-homoserine lactone. Rh1R can selectively bind to lactone signalling molecules, for example N-(3-oxododecanoyl)-L-homoserine lactone and N-butyryl-L-homoserine lactone. LsrR can selectively bind to the AI-2 signalling molecule. ComD can selectively bind to competence stimulating peptides signalling molecules. AgrC can selectively bind to small autoinducing peptides (peptide thiolactones). CepR can selectively bind to lactone signalling molecules, for example N-hexanoylhomoserine lactone. LqsS can selectively bind to the lactone signalling molecules, for example 3-hydroxypentadecan-4-one. QseC can selectively bind to the AI-3 signalling molecule. Ro1B can selectively bind to the AI-2 signalling molecule.

If the chosen quorum sensing protein that selectively binds to a signalling molecule is ComD, ArgC, LqsS, QseC, ro1B or LuxP, binding of the bacterial signalling molecule may cause the phosphorylation of a second protein. This second protein may be membrane-bound.

For example, binding of a signalling molecule to ComC can catalyse the phosphorylation of the response regulator protein ComE (ComE). The binding of a signalling molecule to AgrC can catalyse the phosphorylation of the protein ArgA. The binding of a signalling molecule to LqsS can catalyse the phosphorylation of the protein LqsR. The binding of a signalling molecule to QseC can catalyse the phosphorylation of the protein QseB. The binding of a signalling molecule to ro1B can catalyse the phosphorylation of the protein ro1A. The binding of a signalling molecule to LuxP can catalyse the phosphorylation of the protein LuxQ.

Phosphorylation of the second protein is then what regulates the promoter upstream of the nucleic acid coding for a marker protein.

Hence, if the cell-free expression system comprises the protein ComD, ArgC, LqsS, QseC, ro1B or LuxP or a nucleic acid coding for the protein ComD, ArgC, LqsS, QseC, ro1B or LuxP, the cell-free expression system may further comprise an exogenous second protein or a exogenous nucleic acid coding for a second protein that is phosphorylated when the quorum sensing protein binds to its respective cognate bacterial signalling molecule. Such second proteins include ComE, ArgA, LqsR, QseB, ro1A and LuxQ. Furthermore, the cell-free expression system may further comprise exogenous proteins or exogenous nucleic acid or nucleic acids coding for proteins involved in signalling cascades to enable the phosphorylation of the second protein to activate the promoter upstream of the marker protein.

Promoters that can be used in the invention include pLux, pLqsR, the promoter from the bacterial operon ComCDE, the promoter from the bacterial operon ComA, the promoter from the bacterial operon ComX, the promoter from the bacterial operon Agr, the promoter from the bacterial operon Las, the promoter from the bacterial operon Rh1, the promoter from the bacterial operon Cep, the promoter from the bacterial operon Lsr, the promoter from the bacterial operon flhDC, the promoter from the bacterial operon mot and the promoter from the bacterial operon luxPQ.

The promoters may be directly regulated by the quorum sensing protein or they may be indirectly regulated by the quorum sensing protein. If the promoter is directly regulated by the quorum sensing protein, this may be achieved by the selective binding of the quorum sensing protein to its respective cognate bacterial signalling molecule to produce a quorum sensing protein-bacterial signalling molecule conjugate (also know as a transcription factor or a transcriptional regulator). This conjugate then directly binds to the promoter and hence can activate expression of the downstream marker protein. If the promoter is indirectly regulated by the quorum sensing protein, this may be achieved by the selective binding of the quorum sensing protein to its respective cognate bacterial signalling molecule to effect the phosphorylation of a second protein. The phosphorylated second protein can then regulate the promoter either by binding to the promoter or by a signalling cascade.

The above quorum sensing proteins, second proteins and promoters are merely presented as examples. It would be apparent to one of skill in the art that the cell-free expression system of the invention can be tailored to include proteins or nucleic acids coding for a proteins or promoters other than those listed above, depending on the bacterial signalling molecule being detected.

Example signal transduction pathways that can be exploited by modifying the components of the cell-free expression system are summarised in Table 1.

TABLE 1
Example bacterial signalling molecules their respective cognate binding
protein (quorum sensing protein)

| | Cognate | Second protein | | |
|---|---|---|---|---|
| | Binding | (where | | |
| Signalling Molecule | Protein | applicable) | Promoter | Example Species |
| Competence | ComD | ComE | Promoter from the | Streptococcus |
| Stimulating Peptides | | | operons | pneumonia |
| (CPS) | | | ComCDE, ComA | |
| | | | and ComX | |
| Small autoinducing | AgrC | ArgA | Promoter from the | Methicillin- |
| peptides (AIPs) | | | Agr operon | resistant |
| | | | | Staphylococcus |
| | | | | aureus |
| Lactone based | LasR | — | Promoter from the | Pseudomonas |
| compounds (for | | | Las operon | aeruginosa |
| example N-(3- | | | | |
| oxododecanoyl)-L- | | | | |
| homoserine lactone and | | | | |
| N-butyryl-L- | | | | |
| homoserine lactone) | | | | |
| Lactone based | RhlR | — | Promoter from the | Pseudomonas |
| compounds (for | | | Rhl operon | aeruginosa |
| example N-(3- | | | | |
| oxododecanoyl)-L- | | | | |
| homoserine lactone and | | | | |
| N-butyryl-L | | | | |
| homoserine lactone) | | | | |
| Lactone based | CepR | — | Promoter from the | Burkholderia |
| compounds (for | | | Cep operon | cepacia |
| example N- | | | | |
| hexanoylhomoserine | | | | |
| lactone) | | | | |
| Lactone based | LqsS | LqsR | Promoter pLqsR | Legionella |
| compounds (for | | | | pneumophila |
| example 3- | | | | |
| hydroxypentadecan-4- | | | | |
| one) | | | | |
| Autoinducer-2 (AI-2) | LsrR | — | Promoter from the | Salmonella |
| | | | Lsr operon | typhimurium |
| Autoinducer-3 (AI-3) | QseC | QseB | Promoter from the | Enterohemorrhagic |
| | | | flhDC and mot | Escherichia Coli |
| | | | operon | |
| Autoinducer-2 (AI-2) | rolB | rolA | Promoter pluxSC_{d} | Clostridium |
| | | | | difficile |
| Acyl-homoserine | LuxR | — | Promoter plux | Vibrio fischeri |
| lactone signalling | | | | |
| molecules | | | | |
| Autoinducer-2 (AI-2) | LuxP | LuxQ | Promoter from the | Vibrio harveyi |
| | | | luxPQ operon | |

The cell-free expression system of the invention can comprise a single exogenous nucleic acid which itself comprises a region coding for a quorum sensing protein (wherein the quorum sensing protein is capable of selectively binding to a bacterial signalling molecule) and a promoter operably linked to a nucleic acid sequence coding for a marker protein, wherein the promoter is regulated by the binding of the quorum sensing to a bacterial signalling molecule.

Alternatively, the cell-free expression system of the invention can comprise two different nucleic acids. The first nucleic acid may comprise a region coding for a quorum sensing protein, wherein the quorum sensing protein is capable of selectively binding to a bacterial signalling molecule. The second nucleic acid may comprise a promoter operably linked to a nucleic acid sequence coding for a marker protein, wherein the promoter is regulated by the binding of the quorum sensing to a bacterial signalling molecule.

The exogenous protein capable of selectively binding to a bacterial signalling molecule can be present in the form of soluble protein. The exogenous nucleic acid coding for a quorum sensing protein capable of selectively binding to a bacterial signalling molecule can be present in the form of a deoxyribonucleic acid (incorporated into a plasmid or artificial chromosome or otherwise as required), or in the form of messenger RNA (a ribonucleic acid).

The exogenous nucleic acid coding for a quorum sensing protein capable of selectively binding to a bacterial signalling molecule may also comprise a promoter, for example pTet, T7, SP6 or T3, or any other suitable promoter known to a person of skill in the art.

The nucleic acid or nucleic acids comprising components of the cell-free expression system can be present in the form of plasmids, artificial chromosomes or any other form known by a person of skill in the art to be suitable for cell-free expression systems. The nucleic acid or nucleic acids may comprise a promoter and transcription and translation initiation and termination regions as appropriate.

Cell-free expression systems comprise enzymes and biological machinery required for protein synthesis. These can come from any suitable source, such as defined media or from a cell lysate, such as a bacterial cell lysate, optionally an E. coli lysate. E. coli cell lysates include the E. coli S30 extract, the E. coli S12 extract and the E. coli T7S30 extract (available from Promega UK Ltd., Southampton, United Kindom). The cell extracts can be purchased or made using a population of bacteria, such as E. coli by any means known to a person of skill in the art. Enzymes required for protein synthesis include enzymes required for transcription (for example RNA polymerase), translation (for example peptidyltransferase), post-transcription modification and protein folding.

The cell-free expression systems may also comprise nucleic acids required for protein synthesis, for example ribosomal RNA (rRNA) and transfer RNA (tRNA). The cell-free expression may also comprise initiation factors (for example IF1, IF2, IF3, eIF1, eIF2, eIF3, eIF4 (also known as eIF4F), eIF5 and eIF5B) and elongation factors (for example EF-Tu, EF-Ts, EF-G, eEF- 1 and eEF2).

The cell-free expression system may also comprise an exogenous supply of amino acids. Amino acids include all naturally occurring amino acids, for example alanine, arginine, asparginine, aspartic acid, cysteine, glutamic acid, glutamine, glycine, histidine, isoleucine, leucine, lysine, methionine, phenylalanine, proline, serine, threonine, tryptophan, tyrosine and valine.

The cell-free expression system may also comprise an exogenous supply of nucleotides. These nucleotides can include adenine, guanine, cytosine, thymine and uracil. The nucleotides can be supplied in their deoxy triphosphate state. Thus the exogenous supply of nucleotides can include deoxyadenosine triphosphate (dATP), deoxyguanosine triphosphate (dGTP), deoxycytidine triphosphate (dCTP), deoxythymidine triphosphate (dTTP) and deoxyuridine triphosphate (dUTP).

The cell-free expression system may also comprise an exogenous supply of salts or ions. Such salts or ions can include any salts or ions required for protein synthesis, for example sodium, potassium, calcium, iron, magnesium, ammonium, pyridibium, chloride, nitrate, nitrite, phosphate, sulphate, acetate, carbonate or citrate.

The cell-free expression system may also comprise an exogenous supply of energy generating factors, such as nucleotide triphosphates. Nucleotide triphosphate can include adenosine triphosphate (ATP), guanosine triphosphate (GTP), cytidine triphosphate (CTP), thymidine triphosphate (TTP) and uridine triphosphate (UTP). Other energy generating factors include reduced nicotinamide adenine dinucleotide phosphate (NADPH) and reduced flavin adenine dinucleotide (FADH₂).

Components of the cell-free expression system may be inherently present (endogenous) or may be exogenous.

If the cell-free expression system is obtained from a source containing endogenous genetic content, then that endogenous genetic content may be removed by any means known to a person of skill in the art such that the exogenous genetic material can be preferentially expressed. The cell-free expression system may have an exogenous energy generating system added to it (for example an ATP-generating system) to improve the energy supply.

The cell-free expression system may further comprise buffer solutions or protease inhibitors.

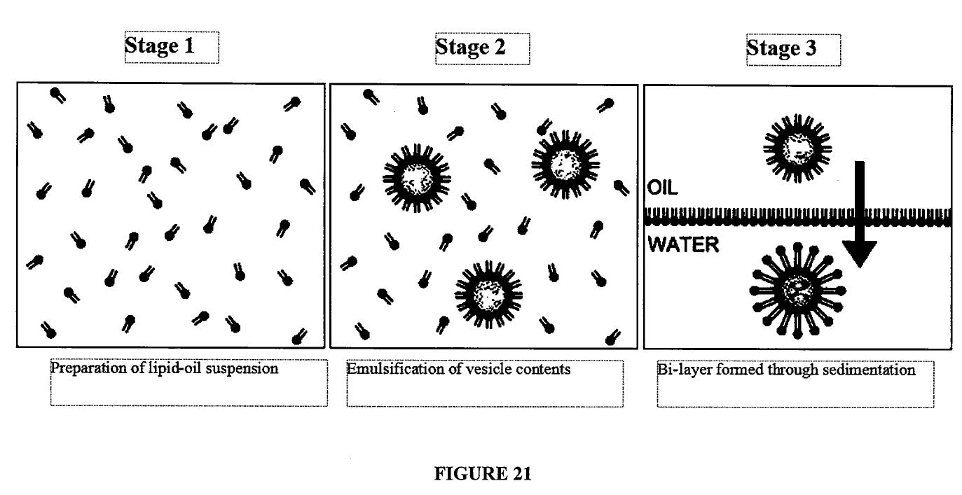

The cell-free expression system can be enclosed in lipid vesicles. This is known as a vesicle-encapsulated cell-free expression system. Vesicles comprising the cell-free expression system can be produced by any means known in the art, for example mixing the cell extract (or components of the cell-free expression system as required) with phospholipids in oil suspension and passing the resulting cell extract particles over an oil/water interface (see FIG. 21).

Alternatively, the cell-free expression system may be a batch-mode cell-free expression system in which transcription and translation is carried out in bulk solution. Alternatively still, the cell-free expression system may be a continous-exchange cell-free expression system in which the transcription and translation is separated from the feeding solution (which comprises any exogenous added components) by a dialysis membrane.

The cell-free expression system of the invention can produce a positive result in the presence of the bacterial signalling molecule being detected after between about 0.1 hours and about 10 hours. More preferably, the cell-free expression system of this embodiment of the invention can produce a positive result in the presence of the signalling molecule being detected after between about 1 hour and 5 hours, between 2 hours and 4 hours, or within about 3 hours.

The use of a cell-free expression system has several advantages over a conventional cell-based protein expression. For example, conditions in a cell-free expression system may be adjusted to promote the correct tertiary and quaternary structure of proteins. They also permit the production of proteins that are not tolerated by a living cell (if, for example, the protein is toxic, proteolytically sensitive or unstable). Additionally, cell-free expression systems permit the production of soluble and functional proteins, whereas cell-based systems can yield insoluble aggregates for many proteins that do not perform their function. Cell-free expression systems have a low mutation rate, since there is no DNA replication, and there is low degradation of the proteins produced (and hence signal conservation is preserved).

There is no concurrent expression of an endogenous genome in cell-free expression systems and hence the genetically engineered device is more energy efficient. Optimum conditions can be maintained by manipulating adjustable parameters (for example buffers can be added to maintain optimum ion concentrations and protease inhibitors can be added to minimize degradation of synthesised proteins). Cell-free expression systems are hence suitable for the expression of a wide variety of proteins and integral membrane proteins insert into membranes may be correctly folded. Furthermore, cell-free expression systems do not contain any living cells so do not represent any potential harm to a patient and furthermore is not restricted by the polices imposed on genetically modified organisms (GMOs).

The cell-free expression system can be applied in the form of a spray, aerosol, ointment, cream, gel, solution, suspension, emulsion or adhesive bandage.

Sprays and aerosols comprising the cell-free expression system for detecting the presence of a bacterial biofilm on a surface can be made by any method known to a person of skill in the art

For example, an aerosol may comprise a canister or bottle with a valve. The canister or bottle comprises the cell-free expression system which may be encapsulated in lipid vesicles. The canister or bottle may further comprise a propellant. Propellants that can be used include an suitable propellants known to a person of skill in the art, including for example propane, n-butane, isobutane, dimethyl ether (DME), methyl ethyl ether, nitrous oxide, carbon dioxide or a hydrofluoroalkane (HFA) (for example HFA 134a (1,1,1,2,-tetrafluoroethane) or HFA 227 (1,1,1,2,3,3,3-heptafluoropropane)), or combinations thereof. The contents of the canister or bottle are kept under pressure such that when the valve is opened, the cell-free expression system is delivered to the surface. As the cell-free expression system escapes the canister or bottle, it is replaced inside the canister or bottle by evaporating propellant. Preferably, the propellant should be miscible with the cell-free expression system.

The aerosol may further comprise a medium in which the cell-free expression system is dissolved or suspended. Such media includes water, phosphate buffered saline (PBS), saline, buffer or any other media known to be suitable by a person of skill in the art.

The canister or bottle can be made of any suitable material known to a person of skill in the art, for example lacquered tinplate (steel with a layer of tin) or aluminium. The canister or bottle can further comprise printed instructions for use.

Aerosols comprising the cell-free expression system can be made by any suitable means known to a person of skill in the art.

A spray comprising the cell-free expression system of the invention can further comprise a bottle or container and an atomiser or spray nozzle. The cell-free expression system may or may not be encapsulated in lipid vesicles. They spray may further comprise a medium in which the cell-free expression system is dissolved or suspended. Such media includes water, phosphate buffered saline (PBS), saline, buffer or any other media known by a person of skill in the art to be suitable.

The bottle or container of the spray can be any bottle or container known by a person of skill in the art to be suitable. For example, such bottles or containers can be made from glass, metal (for example tin, stainless steel or aluminium), plastic or other inert material.

The atomiser or spray nozzle of the spray of this embodiment of this invention permits the conversion of bulk liquid into a mist (collection of droplets) by passing the liquid through a nozzle.

Sprays or aerosols comprising the cell-free expression system of the invention may further comprise preservatives or buffers to prevent decomposition by microbial growth or undesirable chemical changes.

An adhesive bandage (also known as a plaster, a sticking plaster, an ELASTOPLAST®, a BAND-AID® or a small bandage) comprising the cell-free expression system of the invention can further comprise a material on which the cell-free expression system is retained and an adhesive. The adhesive bandage may further comprise a medium in which the cell-free expression system is dissolved or suspended. Such media includes water, phosphate buffered saline (PBS), saline, buffer or any other media known by a person of skill in the art to be suitable. Moreover, the adhesive bandage of this aspect of the invention may further comprise preservatives to prevent decomposition by microbial growth or undesirable chemical changes.

The material of the adhesive bandage may be any material known by a person of skill the art to be suitable, for example woven fabric, plastic or latex rubber.

The adhesive of the adhesive bandage may be any adhesive known by a person of skill the art to be suitable. Preferably the adhesive permits the adhesive bandage to by applied to the surface and easily removed after a suitable time re-expose the surface to ascertain whether or not a bacterial biofilm is present. Example adhesives include an acrylate or methacrylate based pressure sensitive adhesive prepared according to standard industry procedures.

The adhesive is may be a polymerization reaction product of at least two alkyl acrylate or methacrylate ester monomers, at least one ethylenically unsaturated carboxylic acid, at least one vinyl lactam, and most preferably including a crosslinking agent. Examples of suitable alkyl acrylate or methacrylate esters include, but are not limited to, butyl acrylate, ethyl acrylate, 2-ethylhexyl acrylate, isooctyl acrylate, isononyl acrylate, isodecyl acrylate, methyl acrylate, methylbutyl acrylate, 4-methyl-2-pentyl acrylate, sec-butyl acrylate, ethyl methacrylate, isodecyl methacrylate, methyl methacrylate, and the like, or combinations thereof.

The adhesive bandage may comprise an adhesive section where the adhesive is present, and a non-adhesive section where the cell-free expression system is retained.

The adhesive bandage may be covered by an inert material, such as plastic, which protects the bandage from sticking to surfaces other than those being investigated for the presence of a bacterial biofilm. The inert material may also act as a preservative by preventing decomposition by microbial growth or undesirable chemical changes. The inert material is removed prior to the application of the adhesive bandage to the surface to expose the adhesive areas and the non-adhesive area comprising the cell-free expression system.

Creams and ointments comprising the cell-free expression system can be oil-in-water (small droplets of oil dispersed in a continuous aqueous phase) or water-in-oil (small droplets of water dispersed in a continuous oily phase). They may further comprise hydrocarbons (for example hard paraffin or soft paraffin), absorption bases (for example wool fat or beeswax), water soluble bases (for example macrogol 200) or an emulsifier (for example emulsifying wax, cetearyl alcohol, polysorbate 20 or ceteareth 20). Creams and ointments of this aspect of the invention comprising the cell-free expression system may further comprise preservatives to prevent decomposition by microbial growth or undesirable chemical changes.

Solutions and suspensions comprising the cell-free expression system may further comprise a medium in which the cell-free expression system is dissolved or suspended. Such media includes water, phosphate buffered saline (PBS), saline, buffer or any other media known by a person of skill in the art to be suitable. Solutions and suspensions comprising the cell-free expression system may also further comprise preservatives to prevent decomposition by microbial growth or undesirable chemical changes.

In a second aspect of the invention, there is provided an aerosol formulation for detecting the presence of a bacterial biofilm on a surface, comprising a propellant and cell-free expression system, wherein the cell-free expression system comprises:
a) an exogenous quorum sensing protein or an exogenous nucleic acid sequence coding for a quorum sensing protein capable of selectively binding to a bacterial signalling molecule; and
b) an exogenous nucleic acid sequence comprising a promoter operably linked to a nucleic acid sequence coding for a marker protein, wherein the promoter is regulated by the binding of the quorum sensing protein to the bacterial signalling molecule.

In a third aspect of the invention, there is provided an adhesive bandage for detecting the presence of a bacterial biofilm on a surface, comprising a cell-free expression system, wherein the cell-free expression system comprises:
a) an exogenous quorum sensing protein or an exogenous nucleic acid sequence coding for a quorum sensing protein capable of selectively binding to a bacterial signalling molecule; and
b) an exogenous nucleic acid sequence comprising a promoter operably linked to a nucleic acid sequence coding for a marker protein, wherein the promoter is regulated by the binding of the quorum sensing protein to the bacterial signalling molecule.

In a fourth aspect of the invention, there is provided a method of detecting a bacterial biofilm on a surface comprising applying to the surface the cell-free expression system comprising:
a) an exogenous quorum sensing protein or an exogenous nucleic acid sequence coding for a quorum sensing protein capable of selectively binding to a bacterial signalling molecule; and
b) an exogenous nucleic acid sequence comprising a promoter operably linked to a nucleic acid sequence coding for a marker protein, wherein the promoter is regulated by the binding of the quorum sensing protein to the bacterial signalling molecule.

In this aspect of the invention, the cell-free expression system is applied to a surface on which the presence of a bacterial biofilm is being detected. The cell-free expression system may warmed to a temperature sufficient to allow expression of the marker protein to take place (if a bacterial biofilm is present), for example 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 37.5° C., 38° C., 39° C. or 40° C., or any temperature known to be suitable by a person of skill in the art. The cell-free expression system may remain on the surface for a period of time sufficient for the expression of the marker protein. For example, the cell-free expression system may remain on the surface for 0.5, 1, 1.5, 2, 2.5, 3, 3.5, 4, 4.5, 6, 6.5, 7, 7.5, 8, 8.5, 9, 9.5 or 10 hours or any length of time known to be suitable by a person of skill in the art.

To ascertain whether or not a bacterial biofilm is present, it is necessary to investigate whether or not expression of the marker protein has occurred. This may involve carrying out one of any number of assays known to a person of skill in the art to be suitable for detecting the expression of the marker protein in question. For example, if the protein is a fluorescent protein, the surface may be examined under a fluorescent microscope. Alternatively, if the fluorescent protein is visible to the naked eye (for example, in the case of DsRed fluorescent protein), then the expression of the marker protein, and hence the presence of a bacterial biofilm, can be ascertained by simply looking at the surface to which the cell-free expression system was applied.

If the marker protein is a peptide antigen, a person of skill in the art may carry out an immunoassay to detect the expression of the marker protein and hence the presence of a bacterial biofilm. Such immunoassays include ELISA. If the marker protein is an enzyme, a person of skill in the art may carry out an assy to determine the activity of that enzyme and hence the presence of a bacterial biofilm. For example, if the marker protein is luciferase, a person of skill in the art might add luciferin to the cell-free expression system to see if light is produced. If light is produced, then luciferase is present and this is a positive indicator of the presence of a bacterial biofilm.

In one embodiment of the invention, there is provided a method of detecting a bacterial biofilm on a surface comprising applying a cell-free expression system comprising:
a) an exogenous quorum sensing protein or an exogenous nucleic acid sequence coding for a quorum sensing protein capable of selectively binding to a bacterial signalling molecule; and
b) an exogenous nucleic acid sequence comprising a promoter operably linked to a nucleic acid sequence coding for a marker protein, wherein the promoter is regulated by the binding of the quorum sensing protein to the bacterial signalling molecule.

In this embodiment of the invention, the surface may be the external surface of a medical device that is in use. The quorum sensing protein may be LuxR and the promoter may be pLux. The marker protein may be green fluorescent protein.

The medical device may be a catheter. The cell-free expression system may be applied to the external part of the catheter in the form of an adhesive bandage. The cell-free expression system may be warmed to 25° C. After a suitable length of time (for example 3 hours), the adhesive bandage can be removed. The presence or absence of a bacterial biofilm is determined by the presence or absence of fluorescence, respectively. In this embodiment, the detection of acyl homoserine lactone molecules may be achieved by the binding of LuxR to acyl homoserine lactones produced by the bacteria in a biofilm. The LuxR-acyl homoserine lactone conjugate binds to the pLux promoter and in this way stimulates the cell-free expression of green fluorescent protein. Thus, expression of the marker protein is a positive indicator of the presence of a bacterial biofilm.

Preferred features of the first aspect are as for the second and subsequent aspects mutatis mutandis.

The present invention will now be described in details with reference to the following examples which are not to be construed as being limiting on the invention.

### EXAMPLES

### Example 1

### A First Oligonucleotide for use in a Cell-Free Expression System to Detect the Presence of a Biofilm

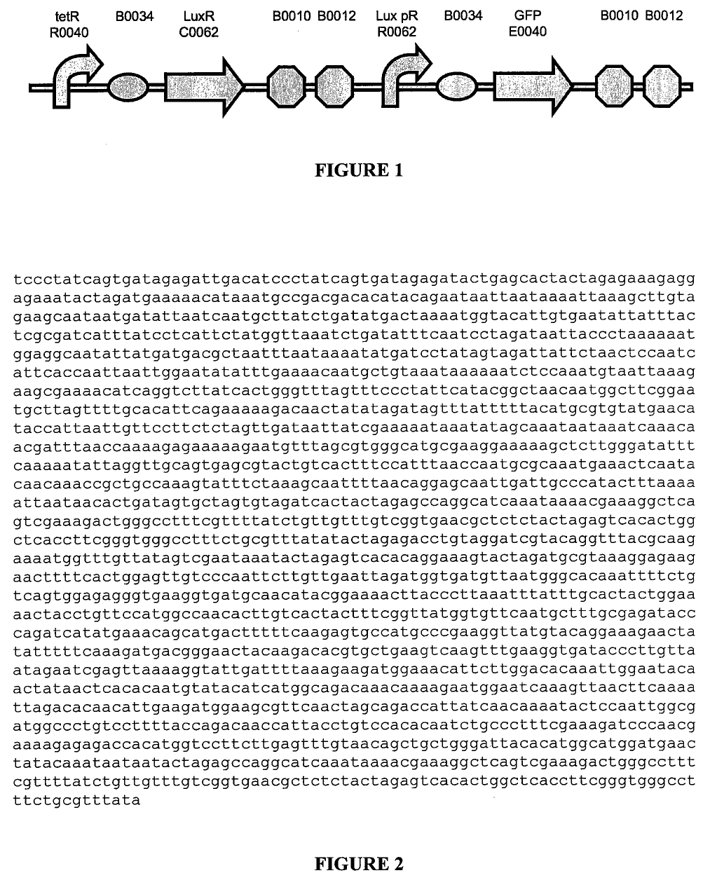

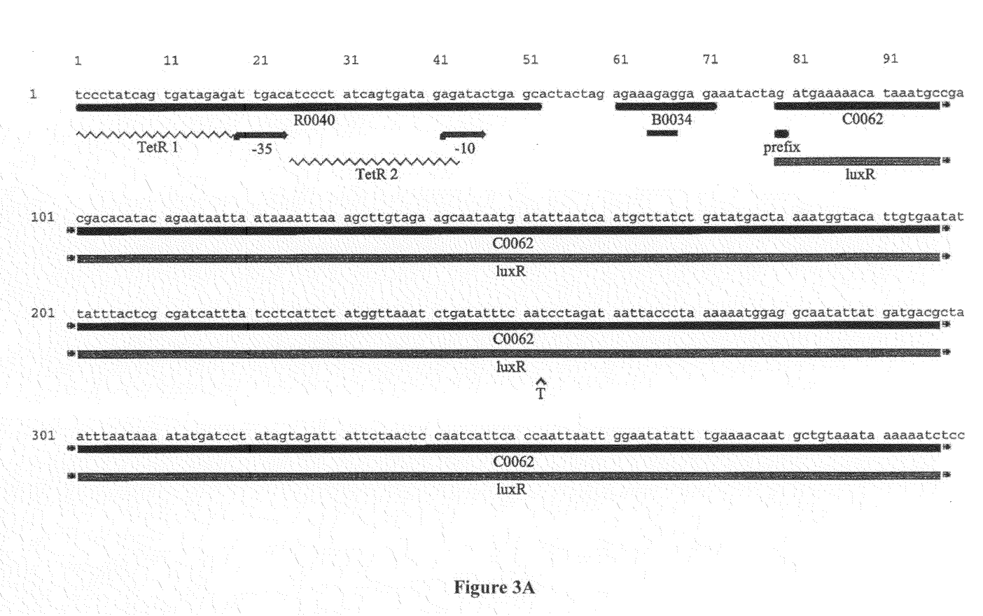

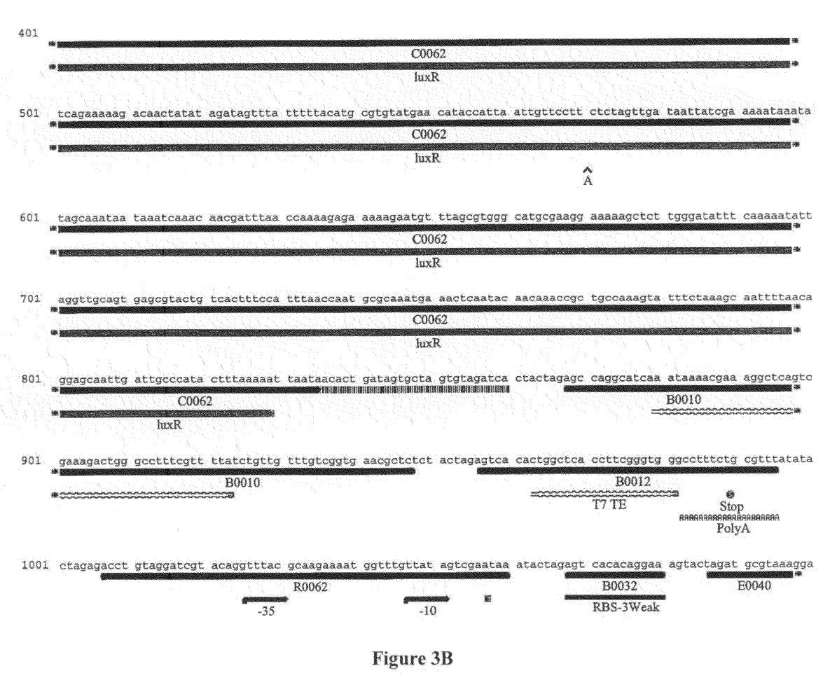

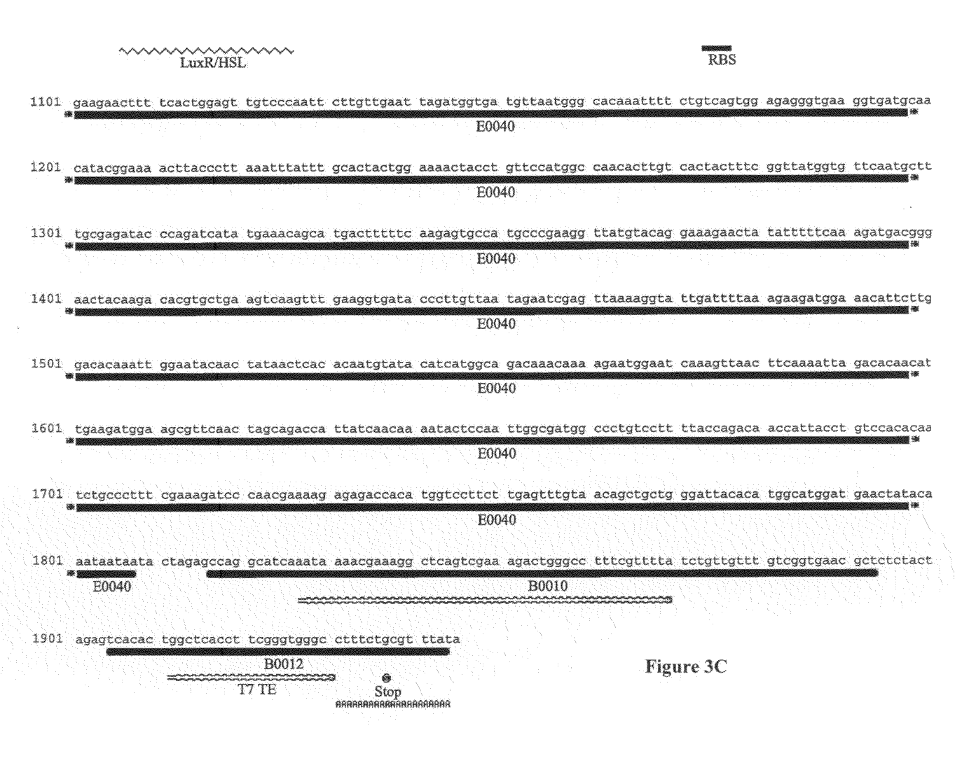

A sequence encoding the luxR gene under the control of the tetR promoter was fused to a sequence encoding the pLux promoter upstream of a sequence encoding a green fluorescent protein. A schematic diagram of the resulting first oligonucleotide (termed construct 1) is given in FIG. 1 and the sequence is given in FIGS. 2 and 3.

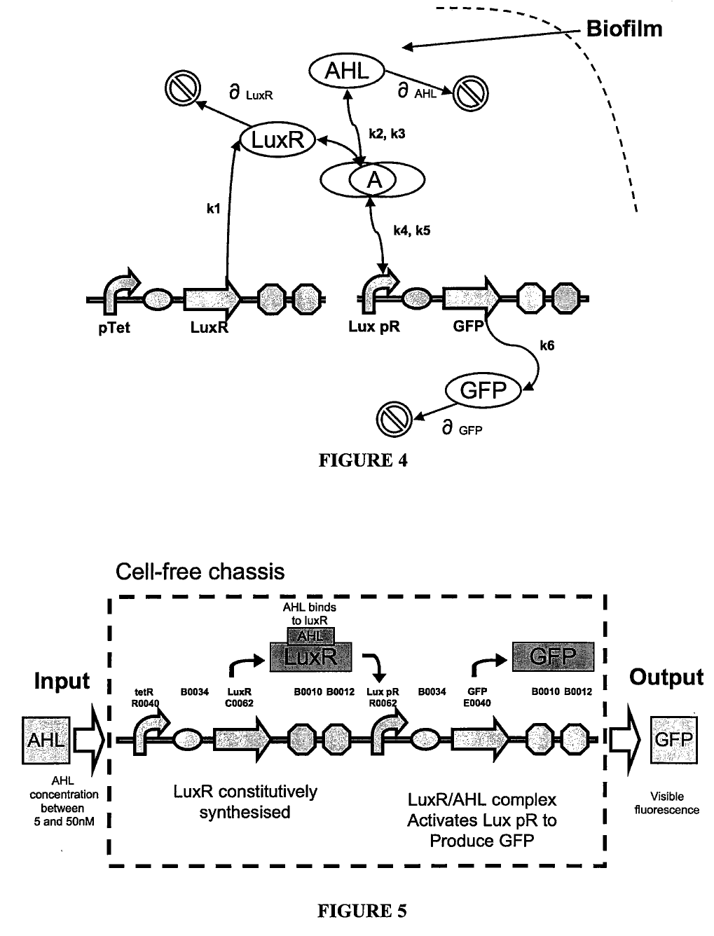

The detection of a biofilm using construct 1 is achieved according to the schematic diagram of FIGS. 4 and 5.

### Example 2

### A Second Oligonucleotide for use in a Cell-Free Expression System to Detect the Presence of a Biofilm

The main feature of this construct is that it does not constitutively express LuxR and therefore enable the initial concentrations of LuxR to be determined manually. LuxR protein is added directly into the cell-free expression.

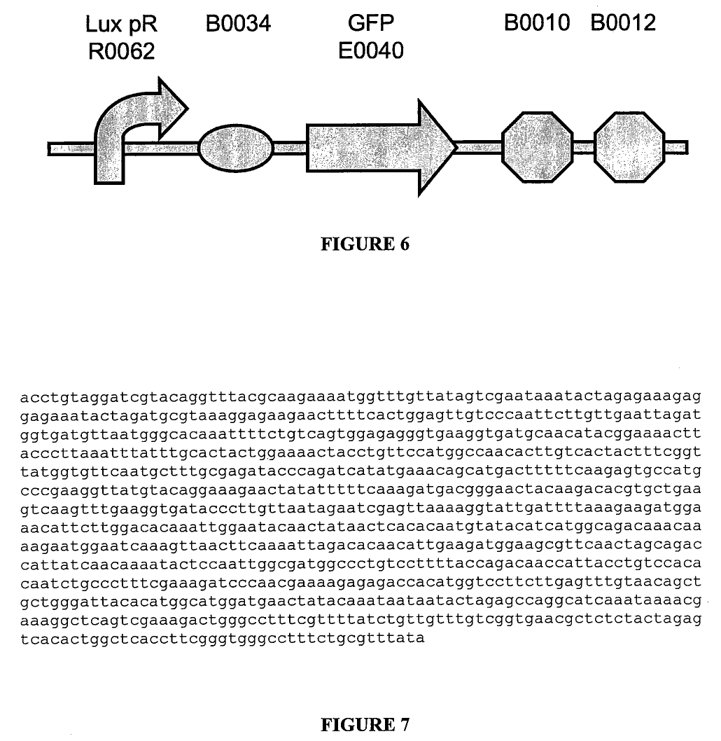

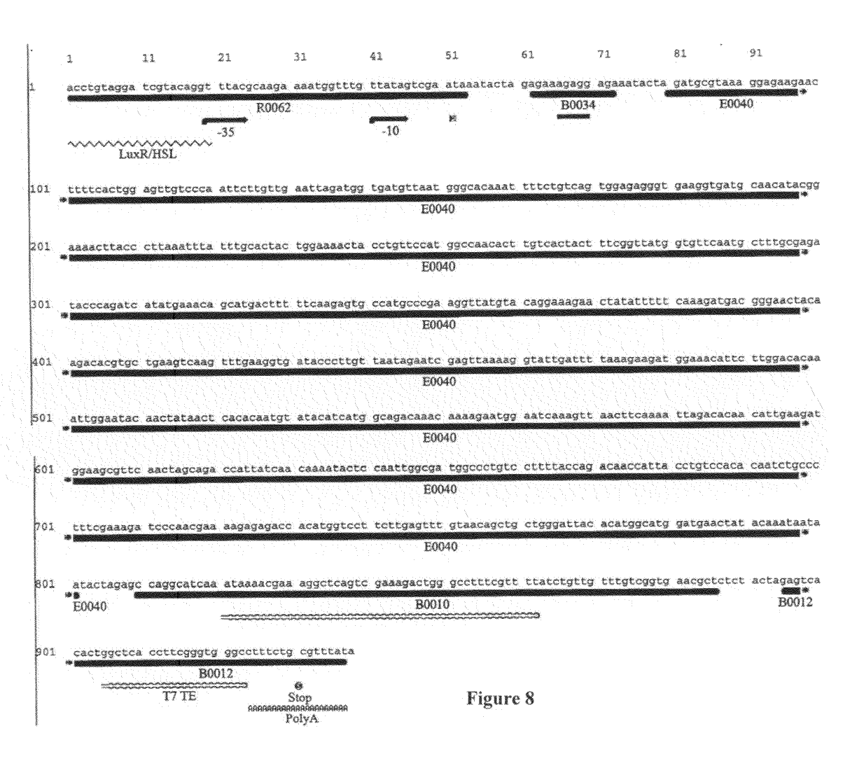

A schematic diagram of the resulting second oligonucleotide (termed construct 2) is given in FIG. 6 and the sequence is given in FIGS. 7 and 8.

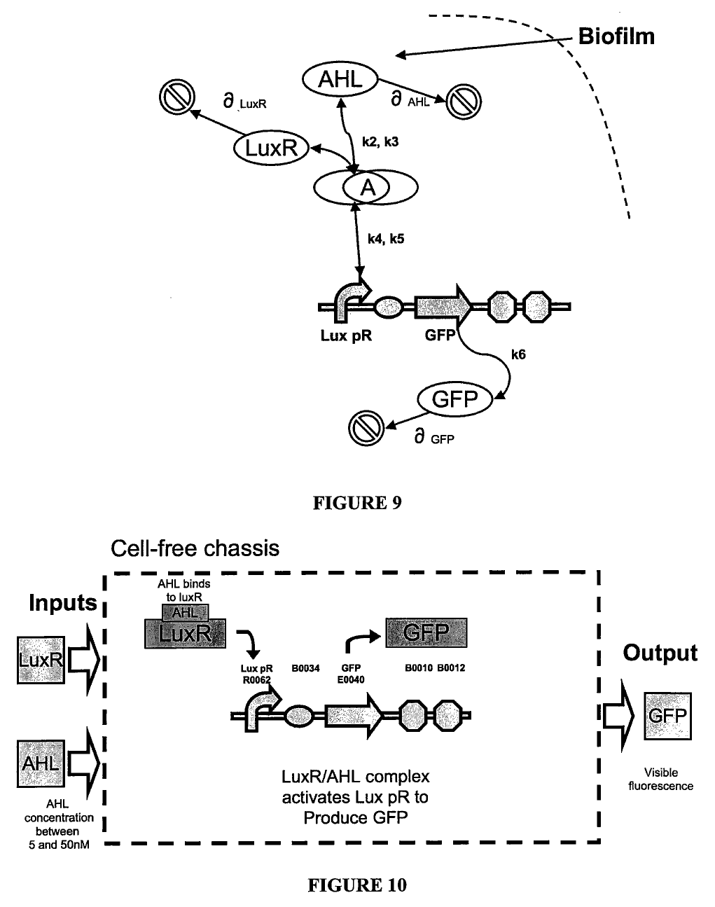

The detection of a biofilm using construct 2 is achieved according to the schematic diagram of FIGS. 9 and 10.

Both designs are based on the following reaction network:
AHL is assumed to diffuse freely “into” the system (a cell-free expression system may come into direct contact with the biofilm).
The target AHL molecule binds with the monomeric protein LuxR.
LuxR is either constitutively produced by construct 1, or directly introduced in purified form, as part of construct 2.
The binding of these two proteins yields the intermediating LuxR-AHL complex, A. k2 and k3 are the kinetic constants of the forward and backward reactions respectively.
The formed transcription factor activates the transcription of the pLux operon, which codes for the relevant reporter protein, GFP. Activation occurs by way of the reversible binding of this transcription factor, A, to the response sequences in the operon.
This leads to recruitment of RNA polymerase and increases the frequency of transcription initiation of the construct gfp gene (strictly forward reaction).

### Example 3

### Modelled Kinetics of the Construct 1

GFP production by construct 1 was modelled at varying initial concentrations of acyl homoserine lactone.

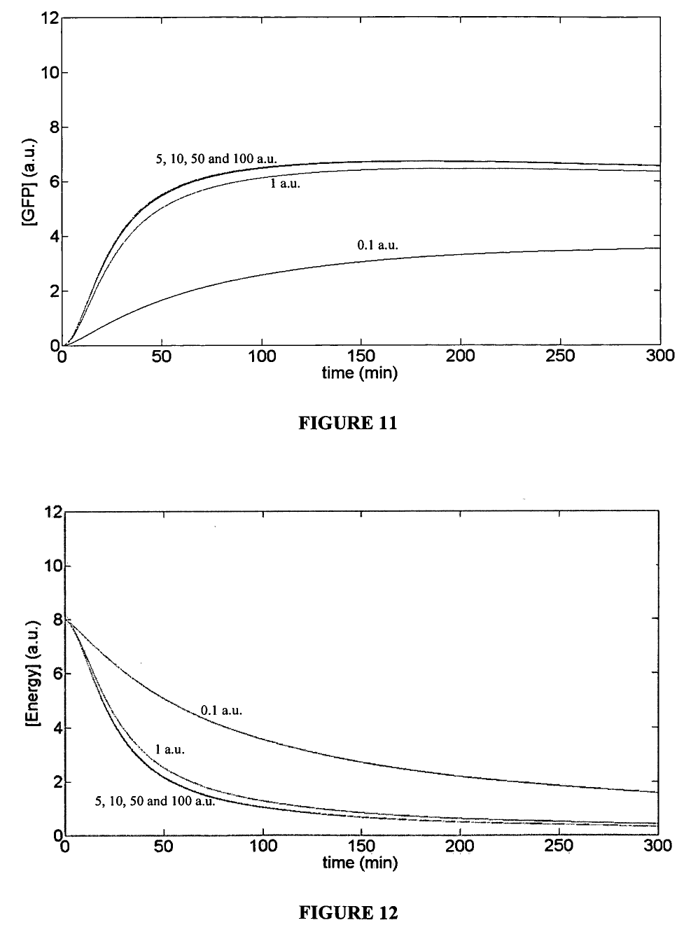

FIGS. 11 and 12 illustrate GFP expression and energy depletion of construct 1, at various initial AHL concentrations ([AHL]s) in arbitrary units (a.u.).

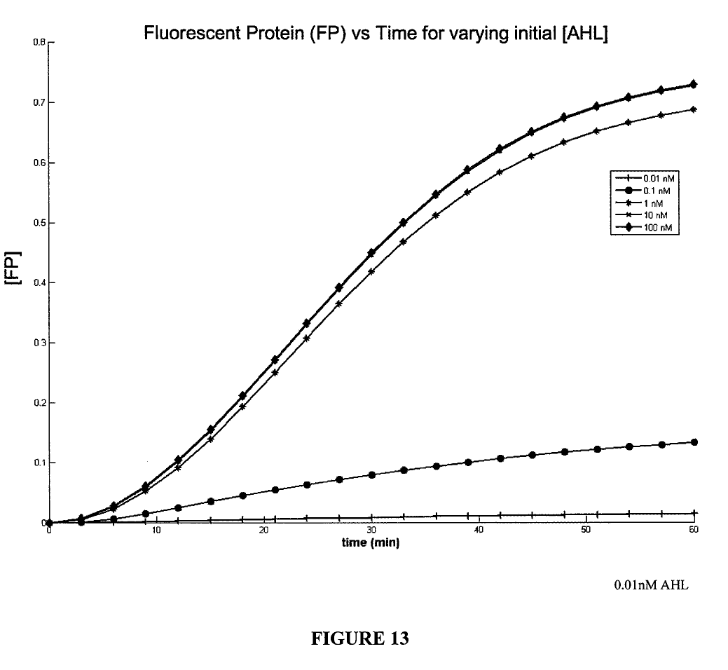

FIG. 13 illustrates GFP expression of construct 1, at various initial AHL concentrations ([AHL]s) in nM.

### Example 4

### Modelled Kinetics of the Construct 2

GFP production by construct 1 was investigated at varying initial concentrations of acyl homoserine lactone.

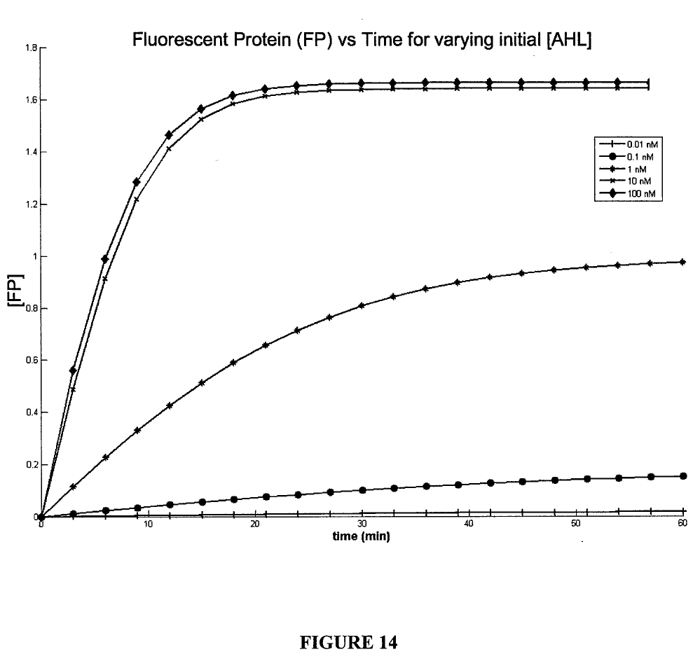

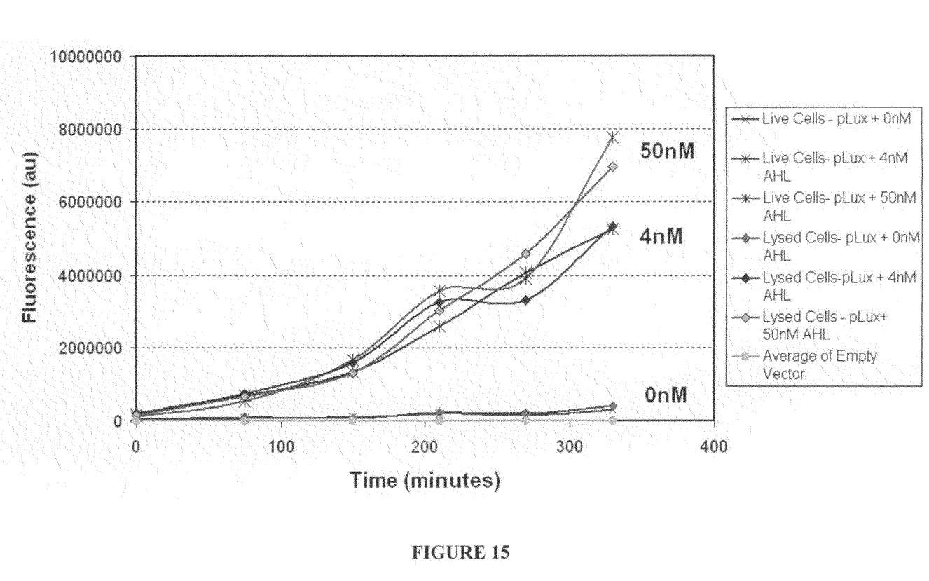

FIG. 14 illustrates GFP expression of construct 2, at various initial AHL concentrations ([AHL]s) in nM. As can be seen by comparing FIGS. 14 and 15, construct 1 takes longer to reach steady state, meaning that over the same time period it reaches a lower maximum value.

Therefore, a cell-free expression system that comprises a protein capable of selectively binding to a bacterial cell signalling molecule would yield faster results and be saturated at higher concentrations that a cell-free expression system that comprises a gene for a protein capable of selectively binding to a bacterial cell signalling molecule.

### Example 5

### Testing the Viability of Construct 1

The construct pTet-LuxR-pLux-GFPmut3b was tested in vivo to determine if the construct works. E. coli BL21 was grown and induced with varying levels of AHL. Two samples were taken every hour from batch cultures, one of these samples was lysed by freeze-thaw and the other whole cells were measured. The results are shown in FIG. 15

FIG. 15 shows that the construct pTet-LuxR-pLux-GFPmut3b works in vivo. The results show that with increasing [AHL] the fluorescence increases, therefore the GFPmut3b produced is increasing with increasing [AHL]. The negative control increases slightly, however remains very low in comparison to the samples.

The difference between the lysed and whole cells does not have a clear pattern, for some samples the lysed cells are higher and in other samples it is lower. This variability is likely to be due to the experimental method, because the lysed and the whole cells where different samples and so error could have been introduced.

The negative control was E. coli BL2I containing an empty vector. Constants were as follows:

Temperature—Cells grown at 37° C.

Volume of Cells sampled—500 μl

Volume Measured—60 ul of PBS buffer

### Protocol to Example 5

### Day 1

### Equipment:

7 ml sterile tubes x4

1.5 ml Eppendorf tube x1

37° C. incubator

Gilson Pipettes p1000 p200 p20

### Reagents:

E. coli BL21; culture containing pTet-LuxR-pLux-GFP

LB medium

Ampicillin stock (50 mg/ml)

AHL stock

### Inoculation of Media:

Inoculate 10 ul of stored parts in individual 2 ml LB medium containing 2 ul of ampicillin. Incubate at 37° C. for overnight in a shaker.

### Preparation of Culture for AHL Induction and GFP Measurement:

From the stock culture of transformed E. coli, dilute the culture so that the OD=0.1.

### Preparation of Diluted Series of AHL:

Add 6 ∞l of AHL stock solution to 600 μl of cell samples, to obtain a final concentration of 1 uM.

### Day 2

### Equipment:

37° C. incubator

Fluorometer+PC

Gilson pipettes 1000 and 200

1 Fluorometer plate (black)

Eppendorf Tubes

Stop watch

### Reagents:

LB medium

E. coli culture with transformed plasmid

10 μM AHL stock

100 μM AHL stock

GFP stock solution

ddH2O

### Preparation of Diluted GFP Standard Solution:

Add 995 ul of ultra pure water an eppendorf tube, together with 5 ul of undiluted GFP standard solution and mix (this gives a 200× dilution to be used as a positive control). Place the tube on ice till it is ready to be used.

### Loading Plate:

1. Transfer 200 μl aliquots of each of the cultures to a flat-bottomed 96 well plate. Three wells to be filled with 200 μl of media to measure the absorbance background. Three further wells to be filled with 200 μl of (media+empty-vector culture) to measure fluorescence background. Standard GFP solution added as a positive control.

2. Add an appropriate amount of stock concentration of AHL to the respective wells as shown.

3. Incubate it at 37° C. for 4 hours.

4. Remove lid and measure in the flourometer (Fluorescence measurements−488 nm excitation filter, 525 nm emission filter, 0.5 seconds, CW lamp energy 12901 units)

5. Repeat the measurement a further two times straight after each other. (This is to test the variability of the machine.)

### Example 6

### Testing the Cell-Free Expression System in an E. Coli S30 Extract

### Preparation

### Aims:

Proper experimental amounts for reaction mixtures of S30 E. coli cell extract from Promega UK Ltd, (Southampton, United Kingdom).

### Equipment:

Eppendorf Tubes

Gilson p20,p200,p1000

### Reagents:

Commercial S30 E. coli extract. Including:

175 μl Amino Acid Mixture Minus Cysteine, 1 mM

175 μl Amino Acid Mixture Minus Methionine, 1 mM

175 μl Amino Acid Mixture Minus Leucine, 1 mM

450 μl S30 Extract, Circular (3×150 μl)

750 μl S30 Premix Without Amino Acids

Nuclease Free water ×1 ml

DNA from midiprep/maxiprep

### Protocols:

From the commercially available kit, one reaction mixture comprises of:
2.5 ul of two different types of amino acid mixtures (total volume of 5 ul)
15 ul of S30 Extract, Circular
20 ul of S30 Premix without amino acids

This makes up a total of 40 ul.

Add the appropriate amount of DNA to the reaction mixture. (For these experiments 2 ug and 4 ug worth of DNA were used)

Top up the mixture to 60 ul with nuclease free water.

### Testing In Vitro

### Aims:

To determine if pTet−LuxR−pLux−GFP (construct 1) DNA constructs for Infector Detector expresses in vitro.

### Equipment:

Fluorometer+PC

30° C. incubator

1 Fluorometer plate (black)

Sticky seal tape

Gilson pipettes p200 p20 p10

Eppendorf Tubes

Stopwatch

### Reagents:

Commercial S30 E. coli extract. Including:

175 μl Amino Acid Mixture Minus Cysteine, 1 mM

175 μl Amino Acid Mixture Minus Methionine, 1 mM

175 μl Amino Acid Mixture Minus Leucine, 1 mM

450 μl S30 Extract, Circular (3×150 μl)

75 μl S30 Premix Without Amino Acids

MiiA water×1 ml

GFP solution

Prepared dilutions of AHL to 1 mM

DNA pTet-LuxR-pLux-GFP

### Protocols:

First collect all equipment and reagents and ensure that the fluorometer and that the PC connected has a data collection protocol installed.

Commercial E. coli Cell Extract (available form Promega UK Ltd.): First prepare a complete amino acid mixture for both extract solutions: Add the 5 μl volume of two amino acid minus mixtures into an labelled eppendorf to give a volume of 10 μl. Each amino acid minus mixture is missing one type of amino acid, and so by combining two solutions we are complementing each solution for the missing amino acid. Place eppendorf in a rack on bench.

Commercial E. coli Cell Extract: Take a eppendorf tube and add 5 μl of the E. coli complete amino acid mixture. Then add 20 μl of S30 Premix Without Amino Acid. Then add 15 μl of S30 Extract Circular. Finally add nuclease-Free Water to bring final volume (including DNA volume) to 100 μl, the volume of DNA added will be determined according to the DNA concentration and the volume of the nuclease free water adjusted accordingly. Place the eppendorf tube in a rack on the bench

Carry out step above again to give two eppendorf tubes of prepared commercial E. coli extract.

Vortex the tubes to mix thoroughly and place the 5× eppendorf tubes in the incubator at 30° C.

### Loading Plate:

Begin by loading the in vitro expression system into a well plate.

Before loading in the samples, vortex the tubes for a few seconds to mix the solution. Place the lid on the 96 well plate and put into the incubator at 30° C. for 10 minutes to allow the temperature to equilibrate. Remove from 30° C. incubator and spin-down in centrifuge in plate centrifuge at 2000 rpm for a few seconds. Spin down is the process of bringing down any solution on lid or side of well into the base of the well. Alternatively, tap the top of the lid to bring down any solution to bottom of the well. Remove lid off the 96-well plate and place in the fluorometer. This measurement will give a background fluorescence measurement and can be used as our time zero data.

Then to begin the reaction, add required volume of purified DNA sample to give 2 μg to the correct wells. Be careful not to add to wells that do not need DNA. Add 1 uM of AHL to the required wells, to a maximum volume of 65 ul per well. Place lid back on and place back in the incubator at 30° C. After 5 minutes of incubation measure the fluorescence by repeating procedure 3-4 above. This initial measurement in the first 30 minutes is to find out how fast GFP is being produced. After this initial measurement, the intervals should be reassessed and adjusted accordingly.

### Results

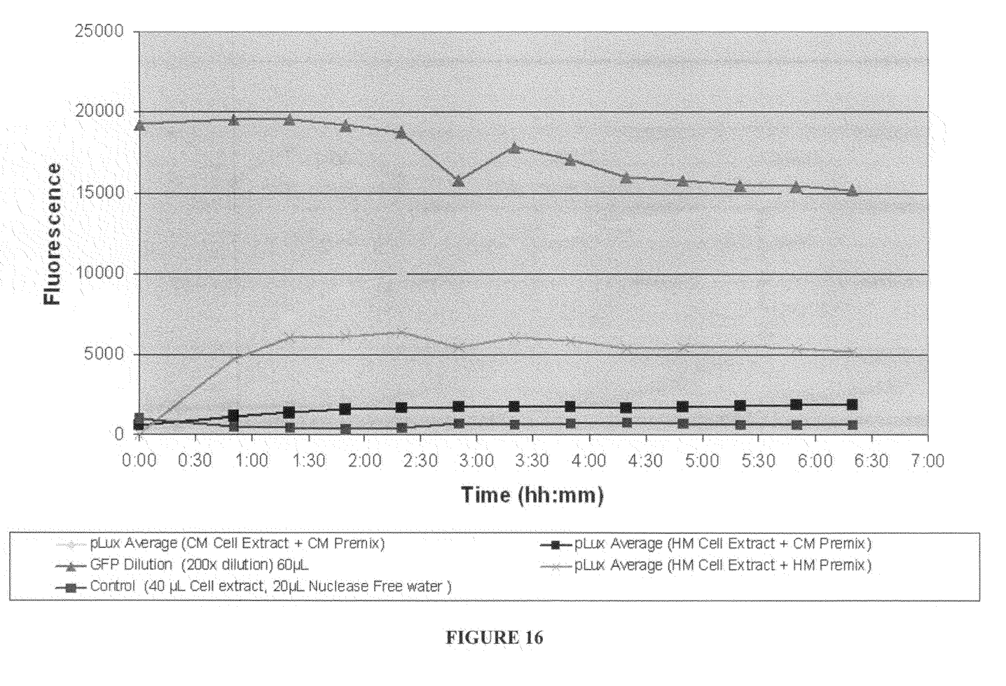

In vitro Testing of pTet-LuxR-pLux-GFP at 25° C.—see FIG. 16.

From FIG. 16, it seems that there is a very strong fluorescent signal observed from the pLux construct using the first combination as its fluorescence has intersected with the positive control after 6 hours.

### Controls:

Positive control—diluted GFP solution of equal volume

Negative control—S30 cell extract of equal volume

### Discussion

FIG. 16 indicates that there was a fair amount of expression of GFP with the pTet-LuxR-pLux-GFP construct, leading to an increase in fluorescence over time. To this, it can be concluded that the construct is working well in vitro.

### Conclusion

pTet-LuxR-pLux-GFP construct works in vitro.

### Example 7

### Optimisation of DNA Concentration

From the initial testing it has been determined that the construct worked in both in vivo and in vitro. The optimal DNA concentration for construct 1 in vitro was determined as follows.

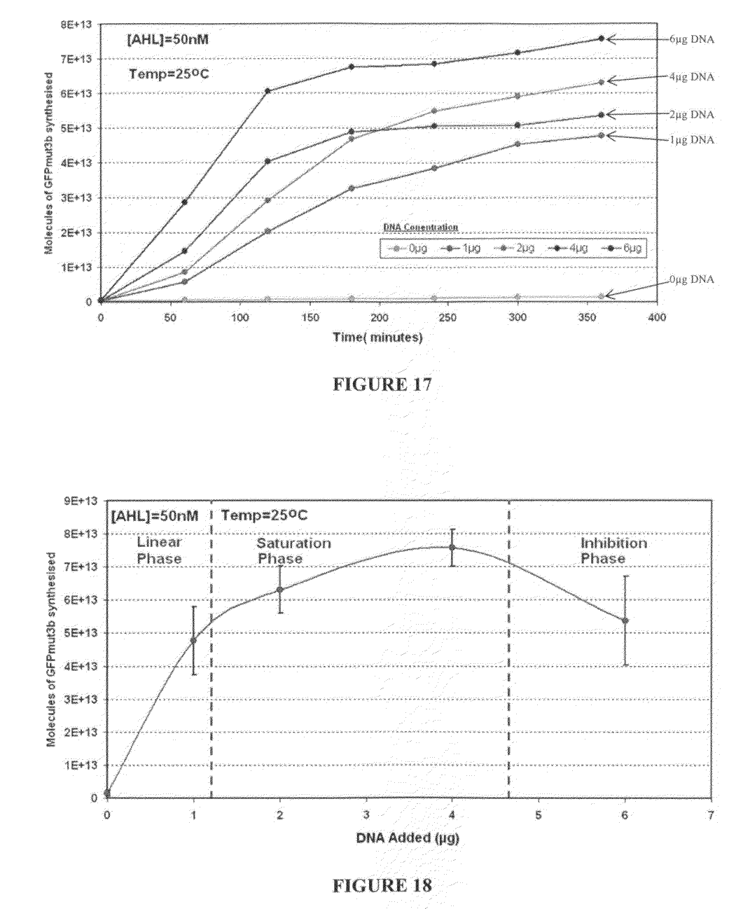

The results are shown in FIGS. 17 and 18. These figures show that the optimum DNA concentration for in vitro is 4 μg for 50 nM AHL. It can be seen that as DNA concentration increases above 4 μg the GFPmut3b molecules synthesised decrease. FIG. 18 can be split into several regions of how the DNA concentration changes the output of GFPmut3b synthesis:

Linear phase—The DNA Concentration is proportional to synthesis of GFP molecules

Saturation phase—The expressional machinery is saturated i.e. RNA polymerases and ribosomes, and so the synthesis is no longer affected by DNA concentration. The maximum synthesis of GFP is at 4 μg.

Inhibition phase—Increasing the DNA concentration actually inhibits the rate of protein synthesis.

### Example 8

### Characterisation of GFP Output for a Range of AHL Inputs

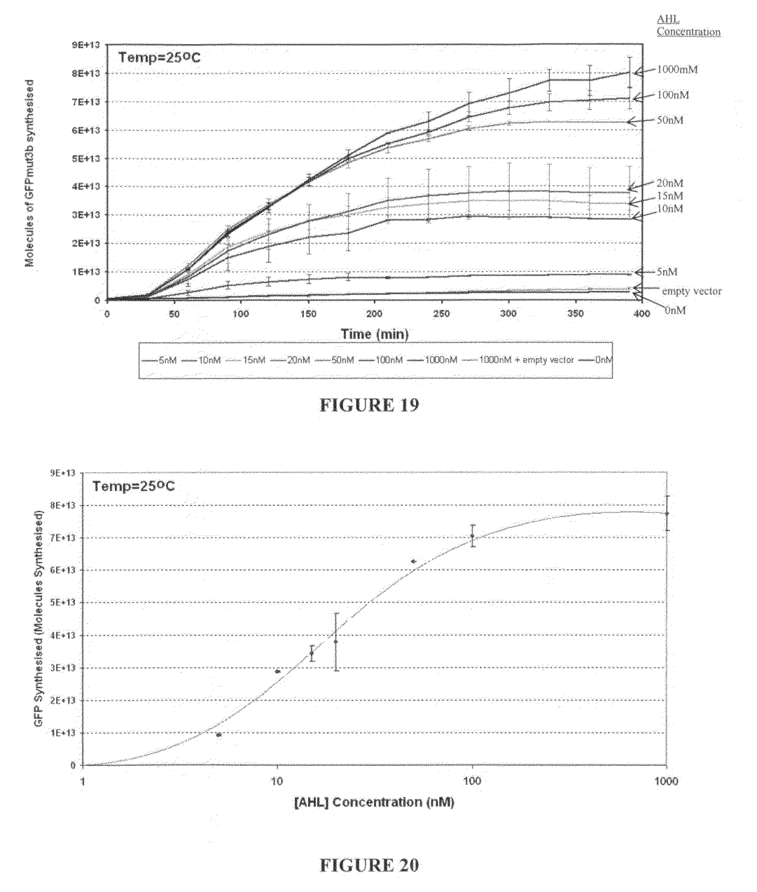

Aim: to test the range 5-50 nM AHL and characterise the output of GFPmut3b for a range of AHL inputs. The results are shown in FIGS. 19 and 20.

FIG. 19 demonstrates that:
The system is functional at 25° C.
The output of GFPmut3b increases with input of AHL.
The system is sensitive to a range of 5-1000 nM AHL.
The GFPmut3b molecules synthesis stops at ˜300 minutes. This could be due to steady state or due to no synthesis of GFPmut3b. It could be steady state because degradation experiments proved degradation is negligible. The time at which synthesis stops is independent of the GFPmut3b molecules produced as all the [AHL] level off at the same time. This shows that the LuxR under the control of pTet is the major source of energy consumption. The pTet is a very strong promoter and is a big consumer of the energy of the chassis. This highlights the advantages of using the construct 2 pLux-GFPmut3b that does not have the energetic burden of producing LuxR under pTet.

### FIG. 20 Demonstrates That:

The shape of the Transfer function shows a linear range of response between 5 nM and 100 nM AHL. This defines the thresholds of response.
The lower threshold of response is the AHL concentration that the construct will respond.
The upper threshold of response is the value of AHL the system is saturated and increasing AHL will not increase the rate of GFP synthesis.

### Example 9

### Vesicle Formulation

For the formation of phospholipid bilayer vesicles, the mineral oil method was chosen. The protocols are based on Engineering Asymmetric Vesicles by Pautot et al. (2003) and Vincent Noireaux et al. (Phys. Biol. (2005) 2(3): 1-8). See accompanying FIG. 21.

The mineral oil method consists of three stages, regardless of composition or equipment:

Preparation of lipid-oil suspension

Emulsification of vesicle contents

Bilayer formation through sedimentation

### Day 1

### Equipment:

Nitrogen tap+plastic tubing

Desiccator connected to a vacuum

100 ml glass bottle

Sonicator with medium-sized probe

Ice bath

25° C. incubator

Pipette+pipette tips (1000 μl)

### Reagents:

10 ml dodecane

12.5 μl 1,2-Dioleoyl-sn-glycero-3-phosphocholine (DOPC) 20 mg/ml in chloroform, ≧99.0%

### Procedure: Preparing the Lipid-Oil Suspension for the Inner Leaflet

Place 125 μl of the 20 mg/ml DOPC solution in a 100 ml glass bottle. With the plastic tubing and 1 ml pipette tip, evaporate the chloroform under nitrogen to obtain a dry, thin lipid film. Put the bottle in a desiccator connected to a vacuum for 1 h. Add 50 ml of mineral oil to reach a final lipid concentration of 0.05 mg/ml. Set the sonicator probe to pulse 1, timer at 30 mins. Put the bottle containing the suspension in the ice bath. Secure the sonicator probe inside the bottle, and set the amplitude to a reading of 10 when it is sonicating. Sonicate the suspension for 30 mins. Leave overnight at 25° C. to ensure that the lipid molecules are fully dispersed in oil.

### Day 2

### Equipment:

Magnetic stirrer

Centrifuge +1-inch glass centrifuge tubes

Pipette+pipette tips (200 μl, 1000 μl)

50 ml glass tube

5 ml syringe

Long 16-gauge stainless steel needle

### Reagents:

10 ml ddH₂O

Tris buffer

NaCl

Reporter

### Procedure: Emulsifying the Aqueous Solution (While the Interface Settles).

Separate about 5 ml of the lipid-oil suspension into a glass container.

This is for the interface preparation. Prepare a 10 ml solution A with 100 mM NaCl and 5 mM Tris buffer at pH 7.4. Prepare solution B by adding a suitable quantity of reporter to 1 ml of solution A. Add 250 μl of solution B to the 45 ml lipid-oil suspension in mineral oil. Gently stir the mixture with a magnetic stir bar for 3 h. Preparing the interface (to be done while the emulsion is mixing)

Place 2 ml of lipid-oil suspension over 3 ml of solution A in a 1-inch-diameter centrifuge tube. Leave for 2-3 h for lipids to achieve the coverage of the interface surface.

To form the vesicles, pour 100 μl of the inverted emulsion over the interface. Centrifuge at 120 g for 10 min.

To collect the vesicles, using a 5 ml syringe with a long 16-gauge stainless steel needle, collect some of solution A. Expel some of the solution to remove all air from the syringe and needle. With the tip of the needle in the aqueous phase, gently expel the solution contained in the syringe. Gently recirculate the buffer several times. Aspirate most of the solution into the syringe, and remove the needle from the solution. Wipe the tip of the needle clean. Unload the vesicle suspension into its final container. (Note: Use optical microscopy to check that the vesicles obtained are not deformed or aggregated.)

### Notes:

Time Required: The lipid-oil suspension preparation takes about 2 h (with a 1 h waiting period 15 min into the procedure), before being left overnight. The remainder of the procedure takes another 4 h, with one 2 h waiting period after an initial 1 h preparation. Total working time in the lab is around 3 hours.

The original protocol uses anhydrous 99:1 dodecane:silicone oil solution instead of mineral oil. The original protocol uses POPC instead of DOPC phospholipids. The original protocol sonicates the suspension in a cleaning sonic bath for 30 min.

Do not use rubber tubing in the nitrogen evaporation. This emits debris into the lipids. This procedure should form around 10⁹ vesicles with 1 μm diameter.

Use of salt in the solution A preparation may require osmolarity considerations.

Use of GFP as a visual signal may require osmolarity considerations.

The reporter in solution B is optional. The vesicles may be visible without it.

The interface should settle for more than 2 h, but less than 3 h. More than 3 h causes the lipids to clump.

### Example 10

### Simulation of Biofilm Detection

It has been found previously that biofilms can be modelled by a gel with pore size of 80nm and fibre thickness of 0.6nm (de Beer et al., Biotechnology and Bioengineering (1997) 53(2):151-158). Agar gel, prepared at a concentration of 2% by weight has approximately these properties (Narayanan et al., J. Phys. (2006) Conf. Ser. 28:83-86) As a preliminary experiment, the viability of agar films and the variability of measurements as a result of their preparation procedure were studied.

Expression of GFP was attempted, using cell extract containing LuxR and the Plux construct, over agar films containing different concentrations of AHL.

Materials and Methods

An agar gel was prepared at a concentration of 2% (by weight) and with AHL dissolved at different concentrations (3 nM, 5 nM, 7 nM, 10 nM). At all times, a water bath at 65° C. was used to prevent the gel from setting before it was loaded into the fluorometer plate. 40 μl of these gel samples were then pipetted into wells in a 96-well fluorometer plate, according to the diagram on the right. Three of the 10 nM AHL agar containing wells were further treated, after setting, with 10 μl of 10× diluted stock GFP solution. Additionally, control wells were set up containing: agar without AHL; agar without AHL and with GFP; GFP only; and blanks (empty wells).

The surface tension between plastic and molten agar solution makes it difficult to pipette and spread the gel at the bottom of the wells. Therefore, at least two wells were loaded with each sample, in order to determine the variability of measurements as a result of the well-loading procedure.

Similarly, three wells were loaded only with 10 μl of GFP solution. The small volume of the solution made it difficult to spread the solution evenly at the bottom of the plate, and it was necessary to test the variability of results due to the location of the GFP solution droplet varying inside the well. Finally, several empty wells were also measured, in order to look at the background fluorescence in them.

The 96-well plate was then loaded onto the fluorometer and fluorescence was measured with a counting time of 0.6 s, with four repeats per well, using a filter for insert wavelength.

Results

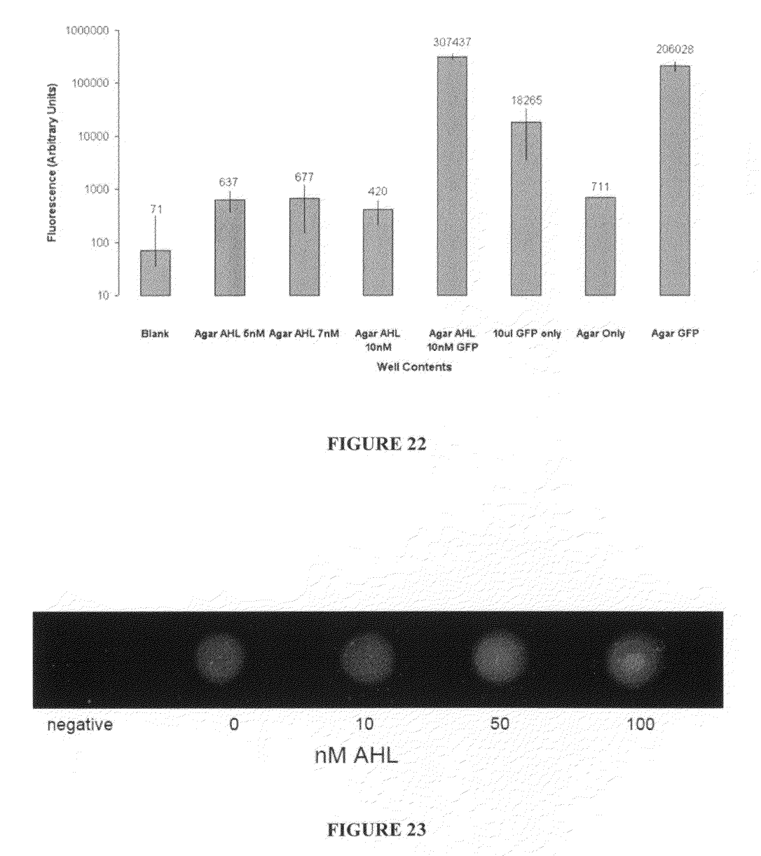

The average of the four readings for each well was taken, and considered as the result for that well. A group (e.g.: blanks) average was then taken from the set of well results for each group. These are shown in FIG. 22. The error bars on the chart are the lowest and highest well averages for that group.

As shown in FIG. 22, wells containing agar only or agar with AHL showed around 5-10 times more fluorescence (around 400 to 700 units) than the empty, or blank, wells (around 71 units). Addition of 10× diluted GFP stock solution to the films increased fluorescence to around 200000-300000 units. The variations in film shape and coverage resulted in differences in fluorescence of up to 50000 units.

FIG. 23 shows the cell-free expression system comprising construct 1 detecting the presence of difference concentrations of AHL on agarose. This clearly demonstrates the ability of the cell-free expression system to detect the presence of bacterial signalling molecules and hence demonstrates its potential uses in biofilm detection.

The disclosures of each patent, patent application and publication cited or described in this document are hereby incorporated herein by reference, in their entirety.

While the foregoing specification has been described with regard to certain preferred embodiments, and many details have been set forth for the purpose of illustration, it will be apparent to those skilled in the art without departing from the spirit and scope of the invention, that the invention may be subject to various modifications and additional embodiments, and that certain of the details described herein can be varied considerably without departing from the basic principles of the invention. Such modifications and additional embodiments are also intended to fall within the scope of the appended claims.

What is claimed is:

1. A cell-free expression system for detecting the presence of a bacterial biofilm on a surface, the system comprising:
a) an exogenous quorum sensing protein or an exogenous nucleic acid sequence coding for a quorum sensing protein capable of selectively binding to a bacterial signalling molecule; and
b) an exogenous nucleic acid sequence comprising a promoter operably linked to a nucleic acid sequence coding for a marker protein, wherein the promoter is regulated by the binding of the quorum sensing protein to the bacterial signalling molecule.

2. The cell-free expression system of claim 1, wherein the quorum sensing protein is selected from the group consisting of LuxR, LuxP, LasR, CepR, Rh1R, LsrR, ComD, AgrC, LqsS, QsecC and ro1B.

3. The cell-free expression system of claim 1, wherein the promoter is selected from the group consisting of pLux, pLqsR, the promoter from the bacterial operon ComCDE, the promoter from the bacterial operon ComA, the promoter from the bacterial operon ComX, the promoter from the bacterial operon Agr, the promoter from the bacterial operon Las, the promoter from the bacterial operon Rh1, the promoter from the bacterial operon Cep, the promoter from the bacterial operon Lsr, the promoter from the bacterial operon flhDC, the promoter from the bacterial operon mot and the promoter from the bacterial operon luxPQ.

4. The cell-free expression system of claim 1, wherein the marker protein is a fluorescent protein, an enzyme or a peptide antigen.

5. The cell-free expression system of claim 4, wherein the fluorescent protein is green fluorescent protein, enhanced green fluorescent protein, blue fluorescent protein, yellow fluorescent protein, cyan fluorescent protein, orange fluorescent protein or red fluorescent protein, or derivatives thereof.

6. The cell-free expression system of claim 5, wherein the expression of the fluorescent protein is visible to the naked eye.

7. The cell-free expression system of claim 6, wherein the red fluorescent protein is DsRed.

8. The cell-free expression system claim 1, wherein the cell-free expression system is a bacterial cell-free expression system.

9. The cell-free expression system of claim 8, wherein the bacterial cell-free expression system is an E. coli cell-free expression system.

10. The cell-free expression system of claim 9, wherein the E. coli cell-free expression system is an E. coli S30 cell-free expression system.

11. The cell-free expression system of claim 1, wherein the surface is the surface of a medical device or the surface of food production equipment.

12. The cell-free expression system of claim 1, wherein the cell-free expression system is applied in the form of a spray, aerosol, ointment, cream, gel, solution, suspension, emulsion or adhesive bandage, or combination thereof.

13. The cell-free expression system of claim 1, wherein the expression of a marker protein is detectable within 3 hours of applying the cell-free expression system.

14. The cell-free expression system of claim 1, wherein the bacterial signalling molecule is selected from the group consisting of a homoserine lactone (HSL), a peptide thiolactone, a furiously borate dieter, a cyclic dipeptide, a competence stimulating peptide (CPS), autoinducer AI-3, bradyoxetin and diffusible signal factor (DSF).

15. An aerosol formulation for detecting the presence of a bacterial biofilm on a surface, the formulation comprising the cell free expression system of claim 1 and a propellant.

16. An adhesive bandage for detecting the presence of a bacterial biofilm on a surface, the bandage comprising the cell free expression system of claim 1.

17. A method of detecting a bacterial biofilm on a surface, the method comprising applying to the surface the cell-free expression system of claim 1.

18. The method of claim 17, wherein presence of a biofilm is indicated by expression of the marker protein.

19. The method of claim 17, wherein the quorum sensing protein is selected from the group consisting of LuxR, LuxP, LasR, CepR, Rh1R, LsrR, ComD, AgrC, LqsS, QsecC and ro1B.

20. The method of claim 17, wherein the promoter is selected from the group consisting of pLux, pLqsR, the promoter from the bacterial operon ComCDE, the promoter from the bacterial operon ComA, the promoter from the bacterial operon ComX, the promoter from the bacterial operon Agr, the promoter from the bacterial operon Las, the promoter from the bacterial operon Rh1, the promoter from the bacterial operon Cep, the promoter from the bacterial operon Lsr, the promoter from the bacterial operon flhDC, the promoter from the bacterial operon mot and the promoter from the bacterial operon luxPQ.

21. The method of claim 17, wherein the marker protein is a fluorescent protein, an enzyme or a peptide antigen.

22. The method of claim 21, wherein the fluorescent protein is green fluorescent protein, enhanced green fluorescent protein, blue fluorescent protein, yellow fluorescent protein, cyan fluorescent protein, orange fluorescent protein or red fluorescent protein, or derivatives thereof.

23. The method of claim 21, wherein the expression of the fluorescent protein is visible to the naked eye.

24. The method of claim 22, wherein the red fluorescent protein is DsRed.

25. The method of claim 17, wherein the cell-free expression system is a bacterial cell-free expression system.

26. The method of claim 25, wherein the bacterial cell-free expression system is an E. coli cell-free expression system.

27. The method of claim 26, wherein the E. coli cell-free expression system is an E. coli S30 cell-free expression system.

28. The method of claim 17, wherein the surface is the surface of a medical device or the surface of food production equipment.

29. The method of claim 17, wherein the cell-free expression system is applied in the form of a spray, aerosol, ointment, cream, gel, solution, suspension, emulsion or adhesive bandage, or combination thereof.

30. The method of claim 17, wherein the expression of a marker protein is detectable within 3 hours of applying the cell-free expression system.

31. The method of claim 17, wherein the bacterial signalling molecule is selected from the group consisting of a homoserine lactone (HSL), a peptide thiolactone, a furiously borate dieter, a cyclic dipeptide, a competence stimulating peptide (CPS), autoinducer AI-3, bradyoxetin and diffusible signal factor (DSF).
